(19) United States
(12) Patent Application Publication
(10) Pub. No.: US 2025/0161691 A1
(43) Pub. Date: May 22, 2025

(54) STIMULATION SYSTEM

(71) Applicant: Maxwell Biomedical, Inc., San Diego, CA (US)

(72) Inventors: Randell WERNETH, San Diego, CA (US); Brandon LEE, San Diego, CA (US); Graydon BEATTY, San Diego, CA (US); Mehdi RAZAVI, Houston, TX (US); Timothy J. CORVI, San Diego, CA (US); R. Maxwell FLAHERTY, Topsfield, MA (US); J. Christopher FLAHERTY, Nottingham, NH (US)

(21) Appl. No.: 18/578,882

(22) PCT Filed: Jul. 13, 2022
(86) PCT No.: PCT/US2022/036926

§ 371 Date: Jan. 12, 2024

Related U.S. Application Data
(60) Provisional application No. 63/221,117, filed on Jul. 13, 2021.
(60) Provisional application No. 63/326,190, filed on Mar. 31, 2022.
(60) Provisional application No. 63/336,517, filed on Apr. 29, 2022.

(51) Int. Cl. A61N 1/365; A61N 1/362; A61N 1/375; A61N 1/378

(52) U.S. Cl. CPC A61N 1/365; A61N 1/3621; A61N 1/37518; A61N 1/3787

(57) ABSTRACT

The invention relates to the use of an electronic element (E) with at least one processor—and memory function (27b), for use as an arithmetic element (6) and/or computer element (6) and/or device element (2a) and/or component element (2a), the electronic element comprising an identification—(1c) and/or authentication function (1c) of a user in a digital network (12) by means of a computer-assisted, at least partly automated and/or autonomously ordered encrypted method (14e), and/or comprising a computer-assisted, at least partly automated and/or autonomously ordered control function (1b), wherein the electronic element (E) consists of at least one hardware element (27) and/or software (26).

### CROSS REFERENCES TO RELATED APPLICATIONS

The present application claims priority to U.S. Provisional Patent Application Ser. No. 63/336,517, entitled “STIMULATION SYSTEM”, filed Apr. 29, 2022, which is hereby incorporated by reference.

The present application claims priority to States Provisional Patent Application Ser. No. 63/326,190, entitled “STIMULATION SYSTEM”, filed Mar. 31, 2022, which is hereby incorporated by reference.

The present application claims priority to United States Provisional Patent Application Ser. No. 63/221,117, entitled “STIMULATION SYSTEM”, filed Jul. 13, 2021, which is hereby incorporated by reference.

The present application, while not claiming priority to, may be related to International PCT Patent Application Serial Number PCT/US2020/049349, entitled “Cardiac Stimulation System”, filed Sep. 4, 2020, Publication Number WO 2021/046313, published Mar. 11, 2021, which claimed priority to U.S. Provisional Application Ser. No. 62/895,655, entitled “Multi-Side Micro Pacing Circuits and Algorithms, Integrated into a Venously Placed Stent Assembly”, filed Sep. 4, 2019, each of which is hereby incorporated by reference.

The present application, while not claiming priority to, may be related to International PCT Patent Application Serial Number PCT/US2021/021467, entitled “Cardiac Pacing Device”, filed Mar. 9, 2021, Publication Number WO 2021/183487, published Sep. 16, 2021 which claims priority to U.S. Provisional Patent Application Ser. No. 62/987,238, entitled “Stent, Mounted and Delivered Wireless, Batteryless Micropacing Chip System”, filed Mar. 9, 2020, each of which is hereby incorporated by reference.

The present application, while not claiming priority to, may be related to International PCT Patent Application Serial Number PCT/US2021/035132, entitled “Pacing and Sensing Devices and Control System”, filed Jun. 1, 2021, Publication Number WO2021/247490, published Dec. 9, 2021, which claims priority to U.S. Provisional Patent Application Ser. No. 63/032,687, entitled “Rechargeable Biomedical Battery Powered Wireless Self-Anchoring Micro-pacing and Sensing Devices and Control System”, filed May 31, 2020, each of which is hereby incorporated by reference.

### FIELD OF THE INVENTIVE CONCEPTS

The present inventive concepts relate generally to stimulation systems, and in particular systems that stimulate tissue of a patient's heart.

### BACKGROUND

The heart is a critical muscle in humans and many other animals that is responsible for circulating blood through the circulatory system. The human heart is made up of four chambers, two upper atria, and two lower ventricles, organized into a left and right pairing of an atrium and a ventricle. In a healthy heart, the chambers contract and relax in a synchronized fashion, referred to as a “beat,” in order to force blood through the network of veins and arteries.

Irregular heartbeats can pose a health risk, and in some cases regular beating can be restored via electrical stimulation. Implantable devices called “pacemakers” are devices which can stimulate the muscle tissue, causing it to contract. By carefully and regularly applying stimulation as needed, normal heart rhythm can be restored.

There is a need for improved systems for treating irregular heartbeats.

### SUMMARY

According to an aspect of the present incentive concepts, a system for providing therapy to a heart of a patient comprises an implantable device configured to be implanted proximate the heart of the patient. The implantable device comprises an anchoring element configured to maintain the position of the implantable device after implantation in the patient, at least one sensing electrode configured to sense electrical activity of the heart, and at least two pacing electrodes configured to deliver electrical stimulation energy to tissue of the heart. The system further comprises a communication device comprising a transceiver configured to transmit energy to the implantable device, and a controller including one or more algorithms. The one or more algorithms are executable to determine parameters of the stimulation energy to be delivered.

In some embodiments, the implantable device further comprises an antenna and a controller.

In some embodiments, the communication device is configured to communicate with the implantable device.

In some embodiments, at least one of the at least two pacing electrodes is implanted on the epicardial surface of the heart.

In some embodiments, at least one of the at least two pacing electrodes is implanted in a cardiac vessel.

In some embodiments, at least one of the at least two pacing electrodes comprises a sensing electrode.

In some embodiments, the system is configured to deliver multi-site pacing via the at least two pacing electrodes, and the multi-site pacing is configured to restore cardiac sinus rhythm. At least one of the at least two pacing electrodes can be positioned proximate the left atrium of the heart. At least one of the at least two pacing electrodes can be positioned proximate the right atrium of the heart. A first electrode of the at least two pacing electrodes can be positioned proximate the right atrium and a second electrode of the at least two pacing electrodes can be positioned proximate the left atrium.

In some embodiments, the system further comprises a clinician device comprising one or more tools configured for percutaneous delivery of the implantable device. The one or more tools can be configured to deliver the implantable device into the pericardial space.

In some embodiments, the implantable device comprises an array of electrodes including the at least two pacing electrodes. The array can comprise a surface area of at least 6.5 cm².

In some embodiments, the implantable device further comprises a power-harvesting antenna.

In some embodiments, the system further comprises a power-harvesting antenna, and the implantable device is connected to the power-harvesting antenna.

In some embodiments, the implantable device further comprises a flexible circuit board.

In some embodiments, the one or more algorithms comprise an algorithm executable to determine the stimulation parameters based on the implantation locations of the at least two pacing electrodes.

In some embodiments, the system further comprises a clinician device comprising one or more catheters configured for transvascular delivery of the implantable device onto the epicardial surface. At least one of the one or more catheters can be constructed and arranged to removably adhere to a vessel wall. The at least one catheter can removably adhere to the vessel wall via a vacuum.

In some embodiments, the implantable device comprises multiple implantable devices. The multiple implantable devices can be each positioned on the epicardial surface. The multiple implantable devices can be configured to wirelessly communicate with each other. The multiple implantable devices can be further configured to communicate with another component of the system.

In some embodiments, the implantable device is further configured to monitor one or more patient parameters. The system can be configured to automatically adjust the therapy provided based on the monitored one or more patient parameters. The system can be configured to monitor at least one patient parameter at a pre-determined time interval. The pre-determined time interval can be programmable by a user of the system. The monitoring of the one or more patient parameters can be initiated by the patient. The one or more patient parameters can comprise at least an ECG, and the system can be configured to adjust the therapy provided based on detection of an arrhythmia via the monitored ECG. The patient parameter can be managed with medication, and the system can be configured to alert the patient to take the medication. The system can further comprise a server, and the system can be configured to transmit patient data to the server.

In some embodiments, at least one of the at least two pacing electrodes comprises an elongated linear electrode.

In some embodiments, the anchoring element comprises at least one barb proximate at least one of the at least two pacing electrodes.

In some embodiments, the system is configured to monitor and/or diagnose sleep apnea of the patient. The system can be configured to monitor and/or diagnose sleep apnea based on monitored EMG and/or ECG signals.

In some embodiments, the implantable device further comprises a fractal antenna. The fractal antenna can comprise multiple self-similar antennae sets, and each set can comprise triangles of a varying size. The fractal antenna can enable energy harvesting over a broad range of frequencies.

In some embodiments, the implantable device further comprises an antenna and two or more electrodes. The antenna can comprise a fractal antenna.

In some embodiments, the system further comprises an external patient device. The external patient device can be configured to transmit power and/or data to the implantable device. The implantable device can further comprise a transceiver, and at least a portion of the transceiver can be implanted sub-dermally proximate the external patient device. Subdermal implantation of the transceiver can be configured to decrease wireless power transfer between the implantable device and the external patient device.

In some embodiments, the system further comprises a clinician device comprising one or more tools configured for transvascular delivery of the implantable device. The one or more tools can be configured to deliver the implantable device into the epicardial space.

In some embodiments, at least one of the at least two pacing electrodes comprises a circular electrode.

In some embodiments, at least one of the at least two pacing electrodes comprises a linear electrode.

In some embodiments, a first of the at least two pacing electrodes comprises a cathodal electrode and a second of the at least two pacing electrodes comprises an anodal electrode, and a bipolar pulse delivered between the first electrode and the second electrode induces an elliptical wave away from the first electrode and causes an anodal block proximate the second electrode. For example, the wave can propagate in all directions away from the first electrode, while simultaneously wrapping around the zone of block surrounding the second electrode.

In some embodiments, the implantable device further comprises a deployable array including one or more filaments.

In some embodiments, the implantable device further comprises an antenna positioned to optimize the geometry and/or orientation of the antenna to maximize the efficiency of power and/or data transfer.

In some embodiments, the implantable device further comprises an antenna configured to be implanted on an endocardial surface in the right atrium.

In some embodiments, at least one of the at least two pacing electrodes comprises an asymmetric profile including a protrusion extending towards cardiac tissue when implanted.

In some embodiments, the implantable device further comprises an insulative sleeve configured to electrically isolate at least one of the at least two pacing electrodes from tissue not intended to receive stimulation energy. Tissue not intended to receive stimulation energy can comprise the phrenic nerve and/or other electrically active thoracic structures.

In some embodiments, at least one of the at least two pacing electrodes is positioned at one or more locations selected from the group consisting of: A1-A6; A2 and A5; A1, A3, and A5; A2, A4, and A6; P1-P5; P1, P2, and P3; P1, P3, and P5; P1 and P2; P1 and P3; P1 and P4; P1 and P5; P2 and P3; P2 and P4; P2 and P5; combinations of groups of one or more A locations and one or more P locations; and combinations thereof.

In some embodiments, at least one of the at least two pacing electrodes is implanted at an anatomical location to allow the left atrium to be adequately and globally captured, such as both temporally and spatially captured.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for diagnosing and/or treating a patient, consistent with the present inventive concepts.

FIG. 2 illustrates an anatomical view of an implantable device implanted proximate a left atrium, consistent with the present inventive concepts.

FIG. 3 illustrates an artistic rendering of an implantable device positioned on the epicardial surface of a left atrium, consistent with the present inventive concepts.

FIGS. 4A and 4B illustrate artistic renderings of implantable devices being deployed from a clinician device onto the epicardial surface of a left atrium, consistent with the present inventive concepts.

FIG. 5 illustrates an artistic rendering of an electrode array positioned on the epicardial surface of a left atrium, consistent with the present inventive concepts.

FIGS. 6A and 6B illustrate various configurations of wires connecting two electrodes of an electrode array, consistent with the present inventive concepts.

FIG. 6C illustrates various configurations of a planar antenna, consistent with the present inventive concepts.

FIG. 6D illustrates an antenna configuration including an extension, consistent with the present inventive concepts.

FIG. 6E illustrates a fractal antenna design and frequency chart, consistent with the present inventive concepts.

FIG. 6F illustrates an anatomic view including a magnification of an implantable device, consistent with the present inventive concepts

FIG. 7 illustrates a method of delivering a device to the epicardial space, consistent with the present inventive concepts.

FIG. 8 illustrates a perspective view of an implantable device comprising a spiral geometry, consistent with the present inventive concepts.

FIGS. 9A and 9B illustrate artistic renderings of implantable devices comprising spiral geometries each positioned on the epicardial surface of a left atrium, consistent with the present inventive concepts.

FIG. 10 illustrates an artistic rendering of an implantable device comprising a spiral geometry being deployed from a clinician device onto the epicardial surface of a left atrium, consistent with the present inventive concepts.

FIGS. 11A-C illustrate various artistic renderings of implantable devices each positioned on the epicardial surface of a left atrium and each comprising an electronic assembly, consistent with the present inventive concepts.

FIG. 12 illustrates a partially transparent anatomic view of the implantation of an implantable device along the epicardial surface, consistent with the present inventive concepts.

FIGS. 13A-C illustrate various anatomic views of the heart showing an implantable device positioned on the epicardial surface, consistent with the present inventive concepts.

FIGS. 14A-G illustrate sequential anatomic images showing the deployment of an implantable device along the epicardial surface, consistent with the present inventive concepts.

FIGS. 15A-C illustrate sectional views of a device positioned within a blood vessel, and a cross sectional view of the device, consistent with the present inventive concepts.

FIG. 16 illustrates an implantable device, consistent with the present inventive concepts.

FIGS. 16A and 16B illustrate various anatomic views showing potential placement locations of multiple implantable devices, consistent with the present inventive concepts.

FIG. 16C illustrates an anatomic view indicating pacing locations, consistent with the present inventive concepts.

FIG. 17 illustrates a method of managing medication, consistent with the present inventive concepts.

FIGS. 18A and 18B illustrate various electrode shapes, consistent with the present inventive concepts.

FIG. 18C illustrates an arrangement of multiple elongate electrodes, consistent with the present inventive concepts.

FIGS. 19A and B illustrate a schematic view of an implantable device and an anatomic view of the heart showing an implantable device positioned on the epicardial surface, consistent with the present inventive concepts.

FIGS. 20A-D illustrate various views of a clinician device, consistent with the present inventive concepts.

FIGS. 21 and 21A illustrate an anatomic view of an implantable device positioned on the epicardial surface and a cross section view of a portion of the device, consistent with the present inventive concepts.

FIGS. 22A and 22B illustrate pairs of front anatomic and partially transparent side anatomic views of a patient wearing an external device, consistent with the present inventive concepts.

FIG. 23 illustrates a method of implanting a device in the pericardial space, consistent with the present inventive concepts.

FIGS. 24A and 24B illustrate anatomic views of an implantable device positioned on the epicardial surface of a left atrium, consistent with the present inventive concepts.

FIG. 25 illustrates a schematic anatomic view of an implantable system, consistent with the present inventive concepts.

FIG. 26 illustrates a schematic view of an implantable device positioned on the epicardial surface of a left atrium, consistent with the present inventive concepts.

FIGS. 27A-C illustrate a side view and two end views of a portion of a device including an electrode with tissue anchors, consistent with the present inventive concepts.

FIG. 27D illustrates a side view of an electrode, consistent with the present inventive concepts.

FIGS. 28A-D illustrate side and perspective views of an implantable device including a tissue anchor, consistent with the present inventive concepts.

FIGS. 29A and 29B illustrate a side view and a sectional anatomic view of an implantable device with anchoring elements, respectively, consistent with the present inventive concepts.

FIGS. 30A and 30B illustrate side views of various anchoring elements, consistent with the present inventive concepts.

FIGS. 31A and 31B illustrate anterior and posterior views, respectively, of a heart, indicating potential electrode implantation locations, consistent with the present inventive concepts.

FIGS. 32A-I illustrate pairs of anterior and posterior anatomic views of the left atrium showing potential electrode implantation location configurations, consistent with the present inventive concepts.

FIG. 33 illustrates a flow chart of a method of treating a patient, consistent with the present inventive concepts.

FIG. 34 illustrates an anatomic view of an implantable device positioned on the epicardial surface of a heart, consistent with the present inventive concepts.

FIG. 35 illustrates a flow chart of steps representing the heart's progression toward heart failure, consistent with the present inventive concepts.

FIG. 36 illustrates an anatomic view of an implantable device positioned on the epicardial surface of a heart, consistent with the present inventive concepts.

FIG. 37 illustrates an anatomic view of a patient wearing external devices, consistent with the present inventive concepts.

FIGS. 38A and 38B illustrate a perspective view of a substrate and a sectional anatomic view of a substrate with a magnified portion, consistent with the present inventive concepts.

FIG. 39 illustrates a side view of an electrode array positioned on a substrate of an implantable device, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.). As used herein, “operably attached”, “operably connected”, and/or “operably coupled” shall refer to electrical, mechanical, communicative, optical, acoustical, fluid, and/or other operable attachments between two or more components.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” when used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component when used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” when used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for providing therapy to a heart of a patient. The system can comprise one or more implantable devices. In some embodiments, the system includes one or more external devices, such as external devices that deliver power and/or data to one or more implantable devices. An implantable device can comprise: an anchor configured to maintain the position of the implantable device; at least one sensor configured to record electrical activity of the heart; and/or one, two, or more pacing electrodes configured to deliver stimulation energy to tissue of the heart, such as to treat an arrhythmia such as atrial fibrillation (AF). The system can include a controller that comprises one or more algorithms, such as an algorithm that initiates and/or adjusts delivery of energy to treat an arrhythmia of the patient.

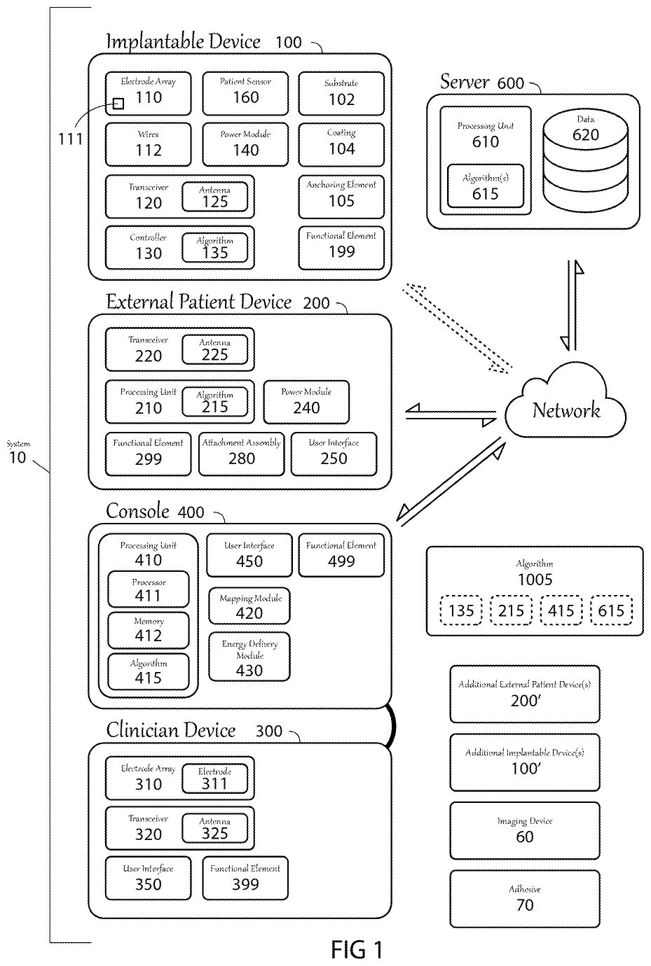

Referring now to FIG. 1, a schematic view of a system for diagnosing and/or treating a patient is illustrated, consistent with the present inventive concepts. System 10 can comprise one or more devices (e.g. devices for a clinician to perform a procedure, devices for a patient to position proximate their body, and/or devices for implantation in the patient) which can be configured to monitor one or more patient parameters, diagnose one or more patient conditions, and/or to treat one or more patient conditions, such as to treat a condition based on one or more patient diagnoses determined by system 10. For example, system 10 can be configured to monitor, diagnose, and/or treat (“treat” herein) atrial fibrillation (AF), a category of abnormally fast and/or “highly irregular” rhythm due to improper electrical activity in the atrial chambers of the heart, such as by monitoring the electrical activity of the patient's heart, and by pacing the muscular tissue in either or both the atrial chambers of the heart to restore sinus rhythm when fibrillation is detected. In another example, system 10 can be configured to treat supraventricular tachycardia (SVT), a category of abnormally fast and/or “regular or quasi-regular” rhythms due to improper electrical activity in the atrial chambers of the heart, such as by monitoring the electrical activity of the patient's heart, and by pacing the muscular tissue in either or both the atrial chambers of the heart to restore sinus rhythm when SVT is detected. In another example, system 10 can be configured to treat atrial tachycardia (AT), a common abnormally fast and regular arrythmia in the category of SVT due to improper electrical activity in the atrial chambers of the heart, such as by monitoring the electrical activity of the patient's heart, and by pacing the muscular tissue in either or both the atrial chambers of the heart to restore sinus rhythm when AT is detected. In another example, system 10 can be configured to treat both the typical and atypical forms of atrial flutter (AFL), which are common abnormally fast and regular arrythmias in the category of SVT due to improper electrical activity in the atrial chambers of the heart, such as by monitoring the electrical activity of the patient's heart, and by pacing the muscular tissue in either or both the atrial chambers of the heart to restore sinus rhythm when AFL is detected. System 10 can be configured to monitor, diagnose, and/or treat other cardiac conditions, such as an arrhythmia, high blood pressure and/or heart failure. In some embodiments, system 10 is configured to both monitor and treat at least one of, or at least two of: an arrhythmia (e.g. atrial fibrillation), high blood pressure, and/or heart failure. System 10 can include one or more devices configured to be implanted, implantable device 100, which can be implanted into the patient for an extended period of time (e.g. at least 1 month, at least 3 months, and/or at least 6 months), such as when implanted by a clinician during a clinical procedure. In some embodiments, implantable device 100 comprises a short-term implant, such as when implantable device 100 is configured to be implanted for no more than 6 months, no more than 3 months, and/or no more than 1 month (e.g. implantable device 100 can be implanted for two or three weeks following cardiac surgery). In some embodiments, ID 100 is configured to be implanted during a cardiac surgical procedure (e.g. an open chest procedure), and ID 100 is configured to be placed on the epicardial surface and adhered thereto, such as with suture, compression, and/or surgical glue.

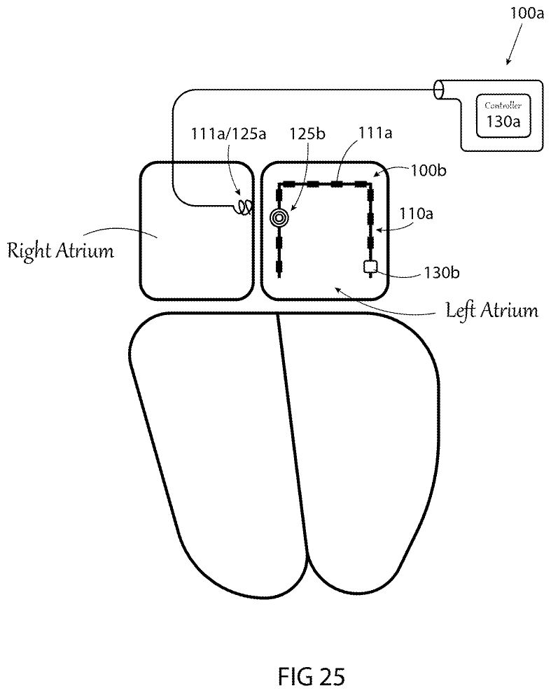

In some embodiments, system 10 comprises one or more externally-placed devices, external patient device 200, which can comprise one or more devices that are configured to monitor, diagnose, and/or treat a patient, such as from one or more locations outside the patient's body. Alternatively or additionally, external patient device 200 (also referred to as EPD 200) can be configured to communicate (e.g. wirelessly communicate) with implantable device 100 (also referred to as ID 100), such as to transfer data between EPD 200 and ID 100, and/or to transfer power from EPD 200 to ID 100. In some embodiments, ID 100 comprises two devices, a first device configured to be implanted proximate the patient's heart, as describe herein, and a second device configured to be implanted at another location under the patient's skin (e.g. subcutaneously). In some embodiments, the second ID 100 (implanted subcutaneously) is configured similar to EPD 200 described herein, such as to transmit power and/or data to the first ID 100 (implanted proximate the patient's heart). In these embodiments, system 10 may include, or may not include, EPD 200. Alternatively or additionally, system 10 may include at least two IDs 100 (such as ID 100a and 100b shown in FIG. 25), where a first ID 100 is configured as EPD 200 and to transmit power and/or data to a second ID 100, and also include an EPD 200, such as when EPD 200 is configured to transmit power to the first ID 100, for example, to recharge a battery of the first ID 100. In some embodiments, one or more IDs 100 and/or EPDs 200 can be operably connected via one or more conduits, not shown, but such as an electrical conduit that is tunneled beneath the skin of the patient (e.g. to connect a subcutaneously implanted ID 100 and/or EPD 200 to an ID 100 implanted proximate the heart).

System 10 can be configured to monitor for and/or to detect irregular or otherwise undesirable (“irregular” or “undesirable” herein) electrical conduction signals or patterns (“patterns” herein) in tissue and/or to deliver energy to the tissue to restore a desirable regular (e.g. healthy) electrical conduction pattern. In some embodiments, system 10 is configured to detect undesirable conduction patterns comprising regular but rapid patterns. System 10 can be configured to monitor the electrical activity of the heart (e.g. conduction patterns proximate the left and/or right atrium of the heart), and to detect the presence of irregular conduction patterns, such as conduction patterns indicative of AF and/or SVT. Additionally or alternatively, system 10 can deliver electrical energy (e.g. pacing pulses) to tissue exhibiting irregular conduction patterns, as well as to tissue surrounding that tissue, to alter the irregular conduction patterns. In some embodiments, system 10 is configured to deliver “multi-site” pacing, where pacing energy is delivered from two, three, four, or more electrodes positioned at different locations, such as different locations proximate the left atrium and/or the right atrium. For example, system 10 can be configured to deliver multi-site left-atrial pacing (e.g. delivery of energy to two left atrial tissue locations) configured to restore sinus rhythm in patients exhibiting irregular conduction patterns. In some embodiments, system 10 is configured to ablate tissue, such as by delivering energy configured to thermally ablate and/or irreversibly electroporate tissue. In these embodiments, system 10 can be further configured to also deliver pacing energy to tissue, such as multi-site pacing energy and/or other pacing energy, such as is described herein.

In some embodiments, system 10 is configured to deliver energy to the patient's heart (e.g. to treat a regular and/or an irregular arrhythmia such as atrial fibrillation, and/or other irregular heartbeat) at a level such that the energy delivery is not perceived by the patient, or at least minimally perceived by the patient (e.g. at a level below a pain threshold). In some embodiments, system 10 delivers energy to the patient as part of a diagnostic study, such as when system 10 is configured to analyze the response of the tissue to the stimulation. In some embodiments, this energy delivery comprises multiple site energy delivery as described herein (e.g. spatially distanced energy delivery). For example, multiple electrodes (e.g. at least three, four, or five electrodes) can be placed on the epicardium for delivery of the stimulation energy. These multiple electrodes can be spatially distributed to gain sufficient coverage of the heart chamber to achieve effective therapy (e.g. to effectively pace-terminate atrial fibrillation and/or other arrhythmia, such as by “gaining control” of the heart chamber by delivering pacing energy from the spatially distributed electrodes). In some embodiments, less than 6 mJ (e.g. less than 5 mJ or less than 4 mJ) of energy can be delivered at each epicardial site (e.g. over a period of approximately one second), such as to terminate atrial fibrillation in the patient.

System 10 can include one or more devices for use by a clinician during a clinical procedure, clinician device 300. Clinician device 300 (also referred to as CD 300) can comprise one or more delivery devices, such as a kit of devices configured to enable the clinician to perform an implantation procedure for implanting ID 100 into the patient. For example, CD 300 can comprise one or more delivery catheters, such as when ID 100 is configured to be implanted during a minimally invasive procedure, such as an interventional procedure performed in a catheterization laboratory (often referred to as a “cath lab”) and/or in another clinical setting (e.g. an outpatient room). For example, CD 300 can comprise one or more tools for percutaneous delivery of ID 100 in the patient's vasculature, and one or more tools for transvascular delivery of ID 100 into locations outside of the patient's vasculature (e.g. into the pericardial space, such as onto the epicardial surface). Alternatively, or additionally, CD 300 can comprise one or more surgical tools (e.g. minimally invasive tools) for surgically implanting ID 100 (e.g. a surgical access kit for use in an operating room and/or any clinical setting in which a minimally invasive procedure can be performed). For example, CD 300 can comprise one or more surgical tools for percutaneous delivery of ID 100 (e.g. a needle-based tool for insertion of ID 100 into the pericardial space). In some embodiments, ID 100 comprises a first geometry where ID 100 is in an undeployed state, such as a geometry comprising a collapsed, folded, or otherwise undeployed geometry configured to allow ease of insertion into the patient. ID 100 can be configured to transition from the first geometry into a second geometry in which ID 100 is in an expanded or otherwise deployed state. In some embodiments, ID 100 is configured to be deployed from a coronary vessel (e.g. through the tissue wall) and implanted along the epicardial surface (e.g. during an interventional procedure), such as is described in detail herein. For example, ID 100 can be deployed from a vessel (e.g. a coronary vessel) selected from the group consisting of: the coronary sinus; the Great Cardiac Vein; the Vein of Marshall; the Azygos vein; a side-branch that anastomoses the coronary sinus, for example, side branches that lie proximate a desired deployment location such as the epicardial surface of the left atrium; and combinations of these. In some embodiments, ID 100 can be configured to be deployed by a robotic delivery device, such as a magnetically-driven robotic device.

In some embodiments, CD 300 includes one or more tools for providing epicardial access (e.g. subxiphoid percutaneous epicardial access), such as to allow a clinician to implant ID 100 on or otherwise proximate an epicardial surface. CD 300 can be configured to prevent (or at least limit the likelihood) of ventricular puncture. CD 300 can be constructed and arranged to enable the clinician to perform a “dry tap” of the epicardial space (e.g. without allowing the needle to penetrate the ventricular tissue). In some embodiments, CD 300 includes one or more devices for positioning a visualizable device, such as a visualizable portion of guidewire or a lead (e.g. a visualizable lead, such as a lead visualizable under fluoroscopy or ultrasound) proximate the lateral margin of the roof of the right atrium (RA). In some embodiments, CD 300 includes a needle and mechanical or other stopping mechanism configured to prevent the needle from advancing into ventricular tissue. After placement, this visualizable device can assist the clinician by providing a visualizable marker indicating the location of the lateral RA boundary. In some embodiments, CD 300 includes one or more devices for positioning a visualizable device proximate the posterior wall of the left atrium (LA). After placement, this visualizable device can assist the clinician by providing a visualizable marker indicating the location of the posterior LA boundary. In some embodiments, CD 300 includes one or more devices for positioning a visualizable device proximate the interatrial septum between the RA and LA. After placement, this visualizable device can assist the clinician by providing a visualizable marker indicating the location of the interatrial septum. In some embodiments, CD 300 includes one or more devices for positioning a visualizable device proximate the apex of the right ventricle (RV). After placement, this visualizable device can assist the clinician by providing a visualizable marker indicating the location of the RV boundary. In some embodiments, CD 300 includes one or more devices for positioning a lead (e.g. a visualizable lead, such as a lead visualizable under fluoroscopy or ultrasound) proximate the apex of the left ventricle (LV). After placement, this lead can assist the clinician by providing a visualizable marker indicating the location of the LV boundary. In some embodiments, system 10 is configured to image the RV, such as with an angiogram or other visualization method (e.g. as provided by imaging device 60 described herein), such as to assist the clinician by providing one or more images that show the border of the RV and the pericardial space. In some embodiments, system 10 is configured to image the LV, such as with an angiogram or other visualization method (e.g. as provided by imaging device 60 described herein), such as to assist the clinician by providing one or more images that show the border of the LV and the pericardial space.

In some embodiments, CD 300 includes a device (e.g. a needle) configured to provide a signal used to identify the pericardial juncture (e.g. by providing a bioimpedance signal). For example, the needle can include an electrode proximate the distal end of the needle. Alternatively or additionally, the needle can comprise an electrically conductive material, and at least a proximal portion of the needle can be insulated such that the distal tip of the needle comprises the electrode. In some embodiments, CD 300 comprises one or more devices configured to be positioned within the coronary sinus, perforate the coronary sinus, and enter the pericardial space. System 10 can comprise one or more visualizable agents, agent 80 shown. In some embodiments, CD 300 is constructed and arranged to inject agent 80 (e.g. a radiopaque material such as contrast) into the pericardial space. For example, device 300 can be configured to inject approximately 10 mL of a contrast-based agent 80 into the pericardial space.

In some embodiments, clinician device 300 comprises a programmer configured to transfer a set of parameters (e.g. a “program”) to one or more of implantable device 100 and/or external patient device 200. In some embodiments, external patient device 200 can transfer programs to implantable device 100. A program can comprise a set of parameters, such as stimulation parameters which implantable device 100 will follow when stimulating the patient, such as described herein. In some embodiments, algorithm 135 is configured to cause implantable device 100 to stimulate the patient based on a program received from EPD 200 and/or clinician device 300.

In some embodiments, CD 300 comprises a stimulator, such as a stimulator similar to a multichannel clinical electrophysiology (EP) laboratory stimulator. CD 300 can be configured to be used during a clinical cardiac EP procedure, such as a procedure that is performed in an EP laboratory. In some embodiments, CD 300 further comprises one or more diagnostic catheters such as catheters configured to be positioned endocardially (e.g. via transvenous access), and/or epicardially (e.g. via pericardial access). The one or more catheters of CD 300 can be connected to a stimulator such that stimulation energy can be delivered to tissue via the catheter (e.g. via one or more electrodes of the catheter).

System 10 can include one or more consoles, console 400 shown. Console 400 can operably connect to CD 300 and can be configured to facilitate one or more processes, energy deliveries, data collections, data analyses, data transfers, signal processing, and/or other functions (“functions” herein) of system 10. In some embodiments, system 10 is constructed and arranged to map electrical activity within the body of a patient (e.g. to map electrical activity of the patient's heart), such as when CD 300 comprises a mapping catheter and console 400 comprises mapping module 420. Mapping module 420 can be configured to record and process mapping signals recorded by CD 300. For example, mapping module 420 can be configured to characterize conduction patterns and/or signal morphologies, such as to classify them (e.g. via algorithm 415 described herein) into arrythmia types, such as AF, AT, AFL, and the like, both typical and atypical. In some embodiments, implantable device 100 and/or EPD 200 are similarly configured to characterize conduction patterns and/or signal morphologies, for example, by processing signals recorded by electrode array 110 (e.g. processing via algorithms 135 and/or 215). In some embodiments, system 10 is constructed and arranged to ablate tissue (e.g. ablate cardiac tissue to treat AF). In these embodiments, console 400 comprises energy delivery module 430. Energy delivery module 430 can be configured to deliver ablative energy to tissue, such as via one or more energy delivery elements (e.g. electrodes, ultrasound transducers, light-emitting elements, and the like) of CD 300. In some embodiments, system 10 is constructed and arranged to stimulate tissue, for example, by delivering stimulation energy via one or more electrodes 311 and/or other energy delivery elements of clinician device 300 (e.g. as described herein). Energy delivery module 430 can be configured to deliver energy in the form of stimulation pulses that stimulate tissue. Energy delivery module 430 can deliver stimulation pulses via any one or more single electrodes 311, and/or via one or more sets (e.g. pairs and/or any quantity) of electrodes 311 at any given instant and/or at any frequency. In some embodiments, stimulation pulses are delivered as a sequence of pulses, such as a sequence of pulses that are delivered simultaneously and/or asynchronously, and/or regularly and/or irregularly. The stimulation pulses can be delivered across a plurality of operably connected electrodes 311, where each electrode 311 can be positioned at prescribed (e.g. clinician and/or system 10 determined, as described herein) locations about a chamber and/or chambers of the heart. These sequences of pulses can be controlled either manually and/or by an algorithm (e.g. algorithm 415 described herein), such as an algorithm that determines the location and the instances in time to deliver stimulation, such as a determination that is based on the measured state of a prescribed chamber's conduction pattern. Console 400 can include processing unit 410, which can be configured to perform one or more functions of console 400 (e.g. as described hereabove). Processing unit 410 can include processor 411, memory 412, and/or algorithm 415, each as shown. In some embodiments, memory 412 stores instructions to perform algorithm 415. Processing unit 410 can be constructed and arranged to execute algorithm 415 and to thereby execute one or more functions of console 400. In some embodiments, console 400 includes one or more user interfaces, user interface 450. In some embodiments, console 400 includes one or more functional elements, functional element 499 shown. Functional element 499 can include one or more sensors and/or transducers.

In some embodiments, console 400 is configured to perform a diagnostic interrogation of the morphology of cardiac activity of the patient, such as to provide a diagnostic interrogation of AF and/or SVT. For example, algorithm 415 can analyze electrical activity of the patient's heart to determine a treatment plan including selection and configuration of one or more components of system 10 to optimize treatment of the patient. In some embodiments, algorithm 415 is configured to process one or more electrograms (e.g. electrograms recorded by system 10 and/or imported into system 10) to produce a 3D model of the electrical activity of at least a portion of the heart. For example, algorithm 415 can produce 3D models that can be displayed (e.g. via user interface 450) to show the electrical conduction patterns and/or conduction timing of a portion of the heart (e.g. one or more chambers of the heart).

The implantation locations of each of the electrodes 111 can be: determined automatically by system 10, determined by a clinician, and/or determined in a semi-automated way based on clinician and system 10 input. In some embodiments, a pacing diagnostic procedure is performed in which energy is delivered by an electrode (e.g. an electrode 311 of clinician device 300, an electrode 111 of ID 100, and/or other electrode) that is positioned in a tissue location temporarily, such as to assess the impact (the pacing impact) of the energy delivery at that location (e.g. an epicardial or other cardiac location). In these embodiments, a set of electrodes 111 implant locations can be determined. In some embodiments, additional criteria can be used to determine the electrode 111 implant locations, such as clinical criteria, anatomical criteria, geometric criteria, criteria derived in simulations, and/or other criteria. These additional criteria can be used cooperatively with the criteria collected in the pacing diagnostic procedure to determine the implant locations for the electrodes 111.

System 10 can include one or more imaging devices, imaging device 60. Imaging device 60 can comprise an imaging device selected from the group consisting of: an X-ray device such as a fluoroscopy device; a CT scanner device; an MRI device; an ultrasound imaging device; and combinations of these.

ID 100 can comprise one or more arrays of functional elements (e.g. sensors and/or transducers), electrode array 110, comprising one, two or more elements, electrodes 111. Electrode array 110 can comprise multiple configurations. Electrode array 110 can be constructed and arranged to be implanted on the epicardial surface of the heart, for example, on the epicardial wall proximate the left atrium. Alternatively or additionally, at least a portion of electrode array 110 (e.g. at least one electrode 111) can be implanted in a vessel (e.g. a coronary vessel), such as the coronary sinus, the Vein of Marshall, the Azygos vein, and/or another vessel proximate the heart. In some embodiments, at least a portion of electrode array 110 is implanted on the endocardial surface, such as within the left atrium or right atrium of the heart, for example, on the septum between the left and right atrium. In some embodiments, at least a portion of electrode array 110 is positioned within the left atrial appendage (e.g. as part of a left atrial appendage closure device). Electrodes 111 can comprise pacing electrodes configured to deliver electrical stimulation energy to patient tissue (e.g. tissue of the heart). Additionally or alternatively, electrodes 111 can comprise sensing electrodes configured to record electrical activity of tissue (e.g. electrical activity of the heart). Electrodes 111 can be configured to deliver electrical stimulation and/or to sense electrical activity in unipolar and/or multipolar (e.g. bipolar or any quantity of multiple electrodes) configurations, such as when two electrodes 111 comprise a pair of electrodes configured to operate in a source and sink arrangement. In some embodiments, electrode array 110 is fixedly attached to one or more flexible membranes, substrate 102. In some embodiments, substrate 102 comprises a single layer membrane. In some embodiments, substrate 102 comprises two or more membrane layers. Substrate 102 can comprise an elastomeric material, for example, a material selected from the group consisting of: poly(lactic-co-glycolic) acid (PLGA); silicone (PDMS); liquid crystal polymers; polyimide; polyurethane (PU); thermoplastic polyurethane (TPU); and combinations of these. In some embodiments, substrate 102 comprises a fabric mesh, such as a polyester mesh. In some embodiments, ID 100 can comprise an anchoring element, such as anchoring element 105 described herein. In some embodiments, substrate 102 comprises a flexible material. In some embodiments, substrate 102 comprises a stretchable material, for example, a material that can stretch at least 5% and/or a material that stretches no more than 200%. In some embodiments, substrate 102 can comprise one or more holes constructed and arranged to cause and/or enhance capillary action of tissue onto substrate 102.

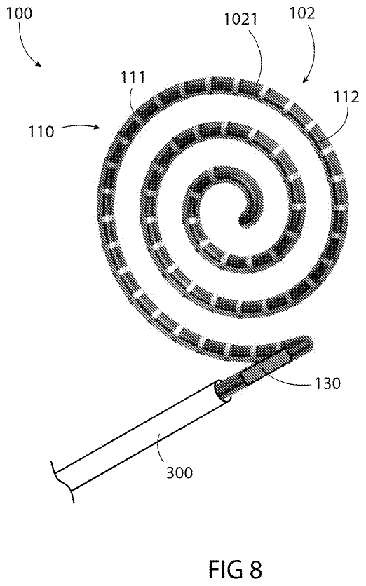

In some embodiments, one or more components of ID 100 (e.g. the components on the outer surfaces of ID 100 which will be exposed to the environment within the body when implanted) comprise biocompatible materials. In some embodiments, one or more components of ID 100 are at least partially encapsulated within substrate 102, for example, electrode array 110 can be positioned between two or more layers of substrate 102. In some embodiments, at least a portion of each electrode 111 of electrode array 110 extends through a layer of substrate 102 (e.g. an outer layer) such that at least a surface portion of electrode 111 is exposed to the body when ID 100 is implanted (e.g. such that ID 100 can be positioned with one or more electrodes 111 (e.g. all electrodes 111) in contact with the epicardial wall). In some embodiments, electrode array 110, and/or other electronic components of ID 100 can comprise one or more elastomeric materials. Alternatively or additionally, electrode array 110, and/or other electronic components of ID 100 can comprise one or more non-elastomeric materials. In some embodiments, substrate 102 comprises an elongate tubular geometry, such as shaft 1021 described in reference to FIG. 8 and otherwise herein.

In some embodiments, electrodes 111 comprise a coating and/or a surface treatment (either or both, “coating” herein), such as a coating that is configured to enhance the recording ability of ID 100 via electrodes 111. For example, electrode 111 can comprise one or more coatings that are configured to increase the surface area of electrodes 111, such as to enhance the recording ability of electrodes 111, such as by lowering the source impedance of electrodes 111. Coatings can reduce the impedance and effects of the half-cell potential that occur from large surface areas of noble metals. A balance of low input impedance and reduced capacitive effects are needed here. Consider that small electrodes are noisy due to high source impedance.

Electrode array 110 can comprise an array surface area (e.g. the surface area defined by the outside boundary of array 110, and/or the convex hull of electrodes 111 of ID 100) of at least 6.5 cm². In some embodiments, electrode array 110 can comprise a surface area greater than or equal to at least 12% of the epicardial surface of the right atrium of the heart and/or the left atrium of the heart. System 10 can comprise multiple implantable devices 100 in various sizes and shapes (e.g. various array 110 sizes), such as when provided in a kit form such that a clinician can select which implantable device 100 of a kit of implantable devices 100 to implant. The selection made can be based on one or more patient parameters, such as the size of the patient's heart (e.g. the size of an atrium and/or a ventricle of the patient's heart). In some embodiments, the size of array 110 of a particular ID 100 is proportional to the amount of tissue through which ID 100 can manipulate the electrical activity of the heart (e.g. to control and/or direct the propagation of cardiac activation of the tissue). In some embodiments, one or more ID 100 can be implanted at a location selected to treat a particular disease or ailment. For example, ID 100 can be implanted proximate the left atrium (e.g. on the epicardial surface) to deliver stimulation energy to treat atrial fibrillation. Alternatively or additionally, ID 100 can be implanted proximate a ventricle of the heart (e.g. on the epicardial surface) to deliver stimulation energy to treat ventricular tachycardia and/or ventricular fibrillation. In some embodiments, at least one electrode 111 is implanted in each chamber of the heart (e.g. at least one ID 100 is implanted in each chamber of the heart), such that system 10 can sense and/or pace from within each chamber.

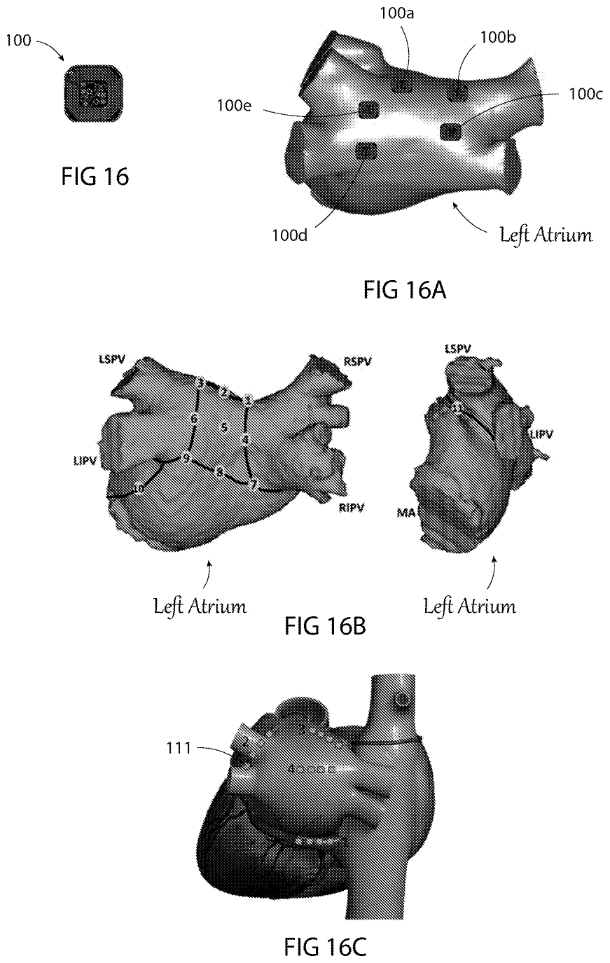

In some embodiments, ID 100 comprises multiple devices, such as at least 5, or at least 10 devices. In these embodiments, multiple implantable devices 100 can be configured to be implanted in a distributed manner, for example, evenly distributed across one or more portions of the epicardial surface. In some embodiments, multiple devices 100 are configured to treat the patient in a coordinated fashion, such as to deliver energy to the cardiac tissue in a pattern based on the location of each individual ID 100 (e.g. relative to each other and/or the cardiac tissue). In some embodiments, ID 100 can comprise multiple devices as described in reference to FIGS. 16, 16A, and 16B herein. In some embodiments, multiple devices 100 are configured to collectively treat a patient with multiple arrhythmias in a coordinated fashion, such as to deliver energy to the right and/or the left atrium to treat AF and/or SVT, and/or to deliver energy to the right and/or the left ventricle to treat other arrhythmias. For example, the multiple devices 100 can each deliver energy as needed (e.g. as determined by a treatment plan of system 10, described herein), such as when the device 100 closest to the source of an arrhythmia is selected to deliver energy to treat that arrhythmia. In some embodiments, at least one of a set of multiple implantable devices 100 can be configured to be implanted in one or more locations selected from the group consisting of: within the right atrium, such as affixed to the endocardial wall of the right atrium; within the left atrium, such as affixed to the endocardial wall of the left atrium; within the left and/or right ventricle; proximate one or more pulmonary veins, such as within and/or partially surrounding a pulmonary vein; on the endocardial surface proximate the left and/or right atrium, the left and/or right ventricle, a pulmonary vein, and/or another anatomic landmark; within a coronary vessel; embedded into cardiac tissue, such as between the endocardial and epicardial surface; and combinations of these.

In some embodiments, one or more conductive portions (e.g. conductive surfaces) of ID 100 (e.g. conductive portions of electrode array 110) are positioned on device 100 to be directed towards tissue to be stimulated when ID 100 is implanted (e.g. directed towards cardiac tissue), and one or more nonconductive portions of ID 100 are positioned on device 100 to be directed toward tissue to be insulated from stimulation energy delivered by ID 100 (e.g. directed toward the pericardium). For example, ID 100 can be configured to be implanted on the epicardial surface with the “bottom” of ID 100 directed towards the epicardial surface, and electrodes 111 can be positioned on the bottom of ID 100 and insulated from the top of ID 100, such as to prevent unintended stimulation of the phrenic nerve, the pericardium, and/or other electrically active thoracic structures. In some embodiments, ID 100 can comprise a cover configured to insulate one or more portions of ID 100 from tissue.

ID 100 can comprise controller 130, which can be configured to perform various functions of ID 100. Controller 130 can comprise a microprocessor, memory, and other components that can be constructed and arranged to control, perform, and/or otherwise enable one or more functions of ID 100. In some embodiments, controller 130 comprises one or more algorithms, algorithm 135 shown. In some embodiments, controller 130 comprises a memory for storing instructions to perform algorithm 135. Controller 130 can be constructed and arranged to execute algorithm 135 and to thereby execute one or more functions of ID 100. In some embodiments, each electrode 111 of electrode array 110 is independently addressable (e.g. electrically connected to at least two wires, such as ground and power or data, between each electrode and controller 130), such that signals (e.g. data and/or power) can be transmitted between controller 130 and each electrode 111 individually or collectively. Alternatively or additionally, controller 130 and/or electrode array 110 can be configured in a multiplexed arrangement, such that each electrode 111 can be individually addressed and/or any combination of electrodes can be addressed via a multiplexing component.

In some embodiments, controller 130 is configured to record electrical activity from one or more electrodes 111 (e.g. one or more electrodes 111 configured as sensing electrodes, such as a pair or other multiple quantity of electrodes configured as one or more sensing electrodes). Additionally or alternatively, controller 130 can be configured to provide stimulation signals to be delivered to the patient via one or more electrodes 111 (e.g. one or more electrodes 111 configured as pacing electrodes, such as a pair or other multiple quantity of electrodes configured as one or more pacing electrodes). In some embodiments, electrode array 110 comprises a set of electrodes 111 configured as pacing electrodes, and a set of electrodes 111 configured as sensing electrodes. In some embodiments, electrode array 110 comprises a first set of electrodes 111 configured as pacing electrodes and a second set of electrodes 111 isolated (e.g. electrically isolated) from the first set of electrodes 111 and configured as sensing electrodes. In some embodiments, electrode array 110 comprises two independent arrays of electrodes, such as a first array of the electrodes 111 configured for pacing and a second array of electrodes 111 (e.g. a second array isolated from the first array) configured for sensing. In some embodiments, the sensing array of electrodes comprises fewer electrodes than the pacing array of electrodes. The second, sensing array of electrodes can be positioned proximate the geometric center of ID 100, and/or within the perimeter of the first array (e.g. when the first array comprises an opening in the center). Alternatively or additionally, controller 130 can be configured to alternate between pacing and sensing from an electrode 111 of electrode array 110 (e.g. in a multiplexed arrangement). In some embodiments, controller 130 is configured to simultaneously sense and pace from a given electrode 111. In some embodiments, multiple electrodes 111 can be multiplexed such as to sense (e.g. record signals) from one electrode 111 relative to a plurality of other electrodes 111 that collectively serve as a sensing reference. For example, the collective reference can be formed by the distance-weighted average of each of the electrodes 111 in the collected-reference (the collective “negative” (−) signal-reference) relative to the one measurement electrode (the one “positive” (+) signal-measurement). Additionally or alternatively, a similar arrangement can be provided for electrodes 111 delivering stimulation energy, such as when stimulation energy is delivered between a set of electrodes 111 (e.g. configured as an anode or a cathode) and a single electrode 111 (e.g. configured as a cathode or anode, respectively).

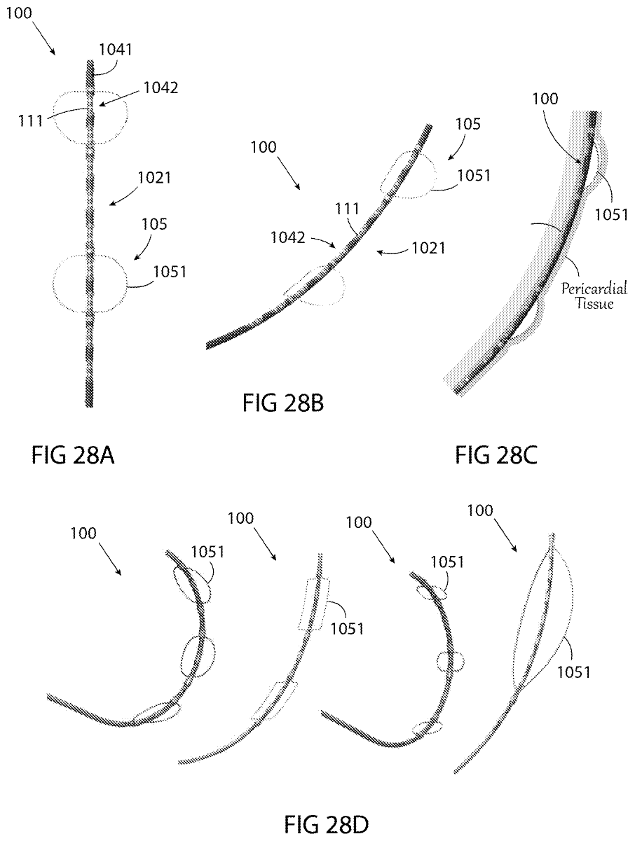

In some embodiments, ID 100 comprises a membrane or other material, coating 104, which can surround at least a portion of the surface of one or more components positioned on and/or within substrate 102. Coating 104 can comprise a biocompatible material, for example, a coating selected from the group consisting of: a silicone (PDMS) coating; a parylene coating; a water-based coating; a resin coating; a chemical coating; a steroidal coating; and combinations of these. Coating 104 can be configured to prevent irritation of the tissue onto which ID 100 is implanted, for example, to prevent an allergic reaction. In some embodiments, coating 104 comprises a bio-adhesive configured to permanently and/or semi-permanently adhere ID 100 to tissue (e.g. to the epicardial wall). For example, coating 104 can comprise a hydrogel (e.g. a hydrogel adhesive). In some embodiments, coating 104 comprises a conductive hydrogel configured to enhance the electrical stimulation energy delivered by ID 100 (e.g. when coating 104 comprises a conductive hydrogel that is positioned on one or more electrodes 111, and/or positioned on at least a portion of a housing of ID 100 that is configured as a return electrode). Alternatively or additionally, system 10 can include adhesive 70, configured to be applied between ID 100 and tissue. In some embodiments, adhesive 70 is electrically conductive. In some embodiments, adhesive 70 comprises a UV activated adhesive. Adhesive 70 can comprise an injectable adhesive, for example, an injectable adhesive comprising a durometer under a threshold (e.g. a sufficiently soft adhesive). Adhesive 70 can comprise a biocompatible adhesive. In some embodiments, coating 104 can comprise a hollow tube, sheath 1041, configured to surround shaft 1021 of ID 100. Sheath 1041 can comprise one or more openings, openings 1042 through which electrodes 111 can contact tissue. Sheath 1041 including openings 1042 can be similar to sheath 1041 described in reference to FIGS. 28A, 28B and otherwise herein.

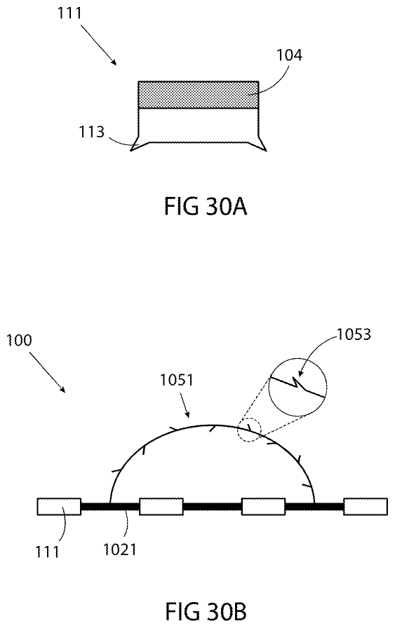

In some embodiments, ID 100 comprises one or more securing and/or stabilizing elements, anchoring element 105. Anchoring element 105 can be configured to secure, affix, stabilize, prevent (or at least limit) migration of, or otherwise prevent or limit unwanted motion of ID 100 (“secure” herein) before, during, and/or after implantation of device 100. In some embodiments, anchoring element 105 comprises a releasable and/or re-securable securing mechanism, such that ID 100 can be repositioned and/or removed (e.g. repositioned by a clinician using clinician device 300). Anchoring element 105 can be configured to interact with an anatomical feature to secure ID 100, such as by pushing against the pericardial sac to force ID 100 onto the epicardial wall. In some embodiments, anchoring element 105 comprises a material configured to promote tissue ingrowth and/or tissue overgrowth, such as to secure ID 100 as tissue growth interacts with anchoring element 105. For example, anchoring element 105 can comprise a fabric mesh. Anchoring element 105 can be similar to anchoring elements described herein in reference to FIGS. 30A and 30B and otherwise herein.

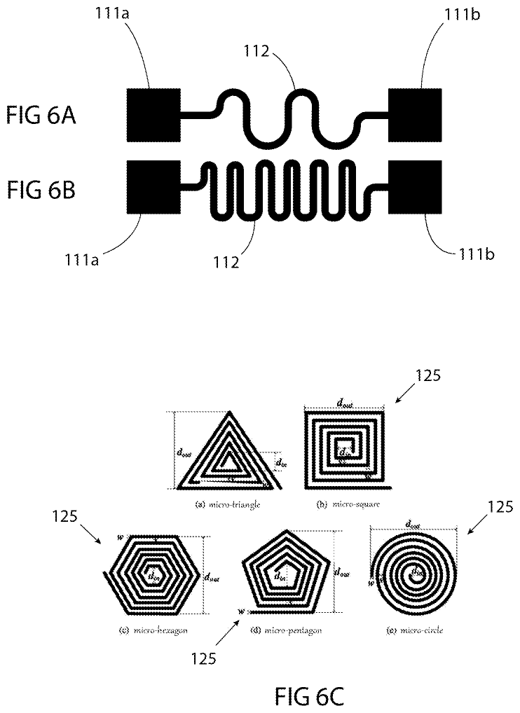

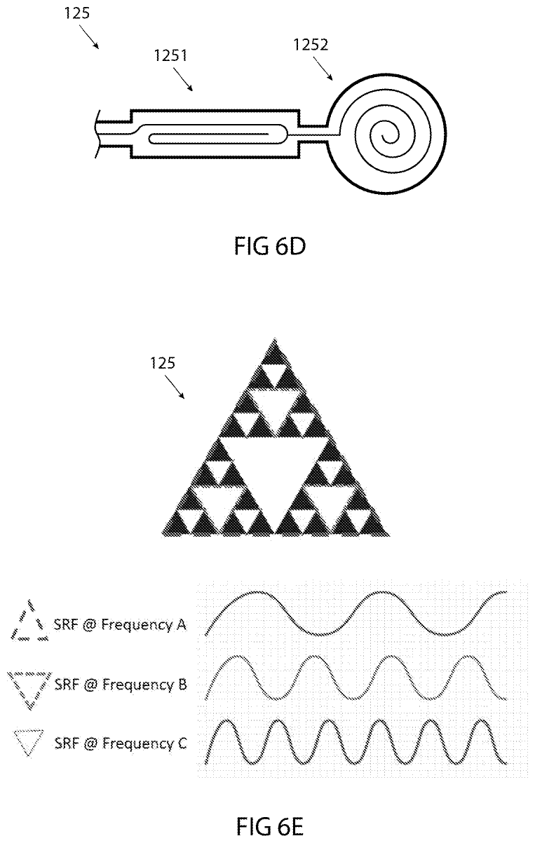

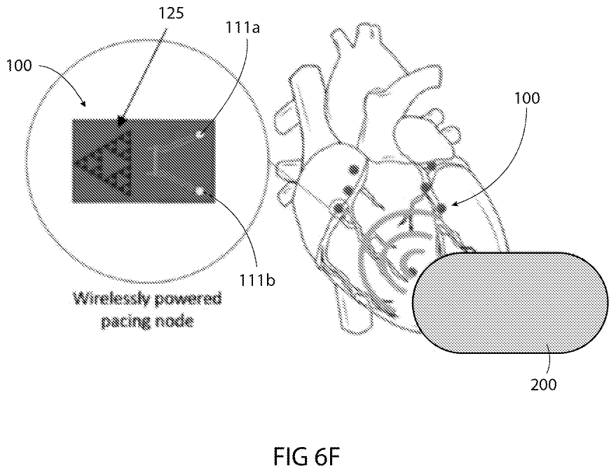

In some embodiments, the various components of ID 100 are interconnected by one or more conduits, wires 112. In some embodiments, wires 112 comprise conductive routing filaments, for example, one or more conductive traces, such as one or more traces within and/or on a circuit board (e.g. a flexible circuit board). In some embodiments, wires 112 comprise traces within and/or on substrate 102. In some embodiments, electrode array 110 comprises one or more wires 112, for example, when electrodes 111 are electrically interconnected by wires 112. Wires 112 comprising conductive routings can each comprise a liquid metal routing, for example, a routing liquid phase eutectic gallium. In some embodiments, conductive traces are applied to substrate 102 (e.g. during a manufacturing process) with methods that include the manipulation of nanoparticles. For example, conductive traces can be formed such that wires 112 comprise nanowires consisting of graphene and/or silver. In some embodiments, wires 112 (configured as conductive traces of substrate 102) comprise a geometry configured to minimize Van der Waals, tensile, compressive, and/or other undesired forces, such as when wires 112 comprise a wavelike geometry (e.g. a sinusoidal geometry). The geometry of wires 112 can be similar to geometries illustrated in FIGS. 6A and 6B described herein. The geometry of wires 112 can be configured such as that wires 112 maintain a high level of conductivity, such when under strain. In some embodiments, wire 112 comprises an insulating coating. In some embodiments, electrode 111 can comprise a portion of wire 112 where the insulation has been removed. In some embodiments, wire 112 is operably attached (e.g. via wired or wireless connecting components) to controller 130, such that controller 130 can perform monitoring and critical analysis and communication via wire 112.

ID 100 can include transceiver 120. Transceiver 120 can be configured to communicate (e.g. wirelessly communicate) with one or more other components of system 10, for example, one or more additional implanted devices 100′, as well as EPD 200, CD 300, console 400, and/or another component of system 10. Transceiver 120 can comprise a receiving and/or transmitting interface, antenna 125. Antenna 125 can be positioned on and/or embedded within substrate 102. In some embodiments, electrode array 110 comprises antenna 125, for example, when wires 112 of electrode array 110 are constructed and arranged to function as an antenna. Antenna 125 can comprise various shapes, for example, antenna 125 can comprise planar micro coils configured in various shapes. Antenna 125 can be configured as illustrated in FIG. 6C described herein. In some embodiments, antenna 125 comprises a wired lead that is placed inside a chamber of the heart. In these embodiments, antenna 125 can be configured to transmit from inside a chamber of the heart to one or more ID 100 placed outside the heart, such as is described in reference to FIG. 25 and otherwise herein. Alternatively or additionally, antenna 125 can comprise a wired lead that is positioned proximate but outside the heart (e.g. on the endocardial surface of the heart). In these embodiments, antenna 125 can be configured to transmit from outside the heart to one or more ID 100 placed within the heart (e.g. within a chamber of the heart).

ID 100 can include power module 140. Power module 140 can include one or more power-generating, power-harvesting, power-storing, power-transferring (e.g. via wireless power transfer) and/or other power-supplying components configured to deliver energy to ID 100. In some embodiments, power module 140 can comprise one or more settings (e.g. stimulation settings and/or other operational settings of system 10), such as clinician configurable settings. Power module 140 can be configured to provide power to one or more components of ID 100. In some embodiments, power module 140 comprises one or more batteries, capacitors, and/or other power-storing devices. In some embodiments, power module 140 includes circuitry that can be configured as a power bypass device. In these embodiments, power module 140 can be configured to transfer power (e.g. power received from an external source) to one or more ID 100.

In some embodiments, power module 140 comprises a solid-state battery, such as a miniature solid-state battery. In some embodiments, power module 140 comprises a rechargeable battery. In some embodiments, ID 100 does not include a battery (i.e. a source of power that is generated by an electrochemical reaction), a “battery-less design” herein, for example, when power module 140 is configured to harvest power (e.g. configured to harvest power transmitted wirelessly from EPD 200), and power module 140 is configured to store and directly provide the harvested power to power the various components of ID 100. Power module 140 can be constructed and arranged to “harvest” power from kinetic motion, for example, from kinetic motion of heart tissue when at least a portion of ID 100 is positioned on and/or within the heart. In some embodiments, power module 140 comprises one or more piezo electric components configured to convert kinetic energy to electrical energy.

In some embodiments, implantable device 100 can comprise patient sensor 160 shown. Patient sensor 160 can comprise one, two or more sensors selected from the group consisting of: an electrical sensor, such as a sensor configured to record an electrogram; a temperature sensor; accelerometer; position sensor; gravimetric sensor; pressure sensor; strain gauge; and combinations of these. System 10 can be configured to monitor one or more patient parameters based on information recorded by patient sensor 160, such as heartbeat, patient position, and/or patient activity.

ID 100 can include one or more functional elements, functional element 199 shown. Functional element 199 can comprise one, two, or more sensors selected from the group consisting of: pressure sensor such as blood pressure sensor; acoustic sensor; respiration sensor; gas sensor such as blood gas sensor; flow sensor such as blood flow sensor; temperature sensor; pH sensor; optical sensor; and combinations of these. In some embodiments, functional element 199 comprises one, two, or more transducers, such as an optical transducer (e.g. an LED).

System 10 can be configured to both monitor one or more patient parameters and to treat the patient based on the monitored parameters (e.g. based on an analysis of the monitored parameters). For example, system 10 can be configured to monitor (e.g. via electrode array 110) and analyze (e.g. via controller 130) electrograms recorded by ID 100, (e.g. unipolar and/or multipolar, for example, bipolar, modes of electrogram recording) and to pace and/or otherwise stimulate tissue if atrial fibrillation (AF) is detected. In some embodiments, system 10 is configured to monitor and/or record one, two, or more of electrophysiological activity, patient temperature, heartbeat information, and/or another patient parameter. In some embodiments, ID 100 is configured to stimulate tissue based on data recorded and/or analyzed by mapping module 420 of console 400. For example, mapping module 420 can be configured to identify irregular conduction patterns within one or more locations of cardiac tissue, as described herein, and to determine a set of stimulation parameters to be delivered by ID 100 to stimulate the tissue to treat (e.g. correct) the irregular conduction patterns.

In some embodiments, electrode array 110 is positioned along (e.g. on and/or within) shaft 1021, such as a shaft 1021 configured in a spiral geometry. Additionally or alternatively, shaft 1021 can comprise a resiliently biased geometry, such as when shaft 1021 is configured in a resiliently biased spiral geometry, such as is described in reference to FIG. 8 and otherwise herein. In some embodiments, substrate 102 comprises a nickel titanium alloy and/or other shape memory material, such as when substrate 102 comprises a shape memory inner layer surrounded by one or more layers of polymers or other flexible materials. In some embodiments, substrate 102 comprises an elongate geometry (e.g. shaft 1021), and the one or more wires 112 extend along (e.g. on the surface and/or within) substrate 102, for example, connecting each electrode 111 to controller 130 positioned at one end of shaft 1021. In some embodiments, controller 130 comprises various other components of ID 100, such as transceiver 120, power module 140, patient sensor 160, and the like.

In some embodiments, wires 112 and/or a portion of substrate 102 are configured as at least a portion of antenna 125, such as when shaft 1021 is configured in a spiral geometry (such as a spiral geometry described herein). For example, substrate 102 can comprise a conductive portion (e.g. an inner core of shaft 1021), such as a portion with a gold conductive core with a nickel titanium cladding, or a nickel titanium core with a platinum iridium cladding. In these embodiments, this conductive core of shaft 1021 can be electrically insulated, such as when the core is surrounded by an insulative material, such as an insulative polymer.

In some embodiments, one or more portions of ID 100 (e.g. one, two, or more components of ID 100) can be bioabsorbable and/or bioresorbable (“bioabsorbable” herein). For example, ID 100 can comprise a device including two or more electrodes 111 operably attached to antenna 125, that is configured to harvest RF energy (e.g. RF energy transmitted from EPD 200) and directly stimulate tissue by providing the harvested energy to electrodes 111 and electrodes 111, antenna 125 and/or the associated electrical traces of ID 100 can comprise a bioabsorbable conductive material, such as tungsten-coated magnesium (W/Mg). ID 100 can comprise one or more other components that comprise bioabsorbable magnesium. In some embodiments, electrodes 111, antenna 125, and/or the associated electrical traces of ID 100 are positioned on and/or within a bioabsorbable patch, such as a bioabsorbable patch configured to be attached to the epicardial surface with bioabsorbable suture.

External patient device 200 (EPD 200) can be constructed and arranged to be worn by the patient, such as when positioned on the skin of the patient (e.g. when EPD 200 is temporarily adhered or otherwise temporarily attached to the patient's skin), and/or when inserted in and/or otherwise attached to the patient's clothing. Alternatively or additionally, EPD 200 can be held against the patient, such as when held against the patient's skin and/or clothing (e.g. by the patient and/or by a patient attachment device). For example, EPD 200 can be configured to be held against the patient, proximate ID 100, while EPD 200 communicates with ID 100 (e.g. for a brief period of time, such as less than 60 seconds). In some embodiments, EPD 200 includes attachment assembly 280. Attachment assembly 280 can include an adhesive, such as an adhesive patch, configured to adhere EPD 200 to the patient's skin for at least 6 hours, such as at least 12 hours, or at least 24 hours (e.g. before the adhesive patch must be replaced). Alternatively or additionally, attachment assembly 280 can comprise a harness, clip, specialized garment, or other non-adhesive based tool for positioning EPD 200 proximate the patient (e.g. proximate the location where ID 100 is implanted in the patient). For example, attachment assembly 280 can comprise a chest strap constructed and arranged to hold EPD 200 over the patient's heart, for example, when ID 100 is implanted onto the epicardial surface of the patient's left atrium. In some embodiments, EPD 200 comprises a device that is implanted subcutaneously or at another internal body location. Alternatively, one or more portions of EPD 200 are implanted in the patient and one or more portions are positioned external to the patient.

EPD 200 can include transceiver 220. Transceiver 220 can be configured to communicate (e.g. wirelessly communicate) with one or more components of system 10, for example, one or more implanted devices 100, and/or one or more additional external patient devices 200′, as well as CD 300, console 400, and/or other components of system 10. Transceiver 220 can comprise a receiving and/or transmitting interface, antenna 225. EPD 200 can be constructed and arranged to transmit power and/or data to one or more implantable devices 100, such as by transmitting a radio frequency (RF) energy from antenna 225, through the skin of the patient, towards ID 100, and ID 100 can be constructed and arranged to harvest the RF energy and/or receive the RF data via antenna 125 (e.g. a power-harvesting antenna). In some embodiments, EPD 200 is constructed and arranged to receive data from one or more implantable devices 100, such as when transceiver 120 is constructed and arranged to transmit RF data to EPD 200.

EPD 200 can include one or more user interfaces, user interface 250 shown. User interface 250 can include one or more user input and/or user output components, for example, one or more: displays, indicators (e.g. LEDs), speakers, buttons, microphones, haptic interfaces, and/or other user interface components. In some embodiments, EPD 200 includes one or more functional elements, functional element 299 shown. Functional element 299 can include one or more sensors and/or transducers. User interface 250 can display a visual representation of the heart chambers (e.g. a digital model) including one or more electrical conduction patterns (e.g. AF conduction patterns and/or sinus rhythm conduction patterns) that are displayed relative to the representation of the heart anatomy. In some embodiments, user interface 250 can display a representation of one or more portions of ID 100 (e.g. one or more electrodes 111) relative to the representation of the heart. In some embodiments, the conduction patterns displayed include pre-treatment and/or post-treatment (e.g. post pacing) conduction patterns. In some embodiments, the conduction patterns are displayed relative to each electrode 111 that is displayed on the representation of the heart. In some embodiments, user interface 250 can display various simulations of conduction patterns resulting from a proposed therapy to be delivered to treat the arrhythmia (e.g. AF) of the patient.

In some embodiments, functional element 299 of EPD 200 comprises one or more sensors that are used to record a patient parameter, such as a patient EEG. For example, functional elements 299 can comprise one, two, or more sensors (e.g. electrodes) that are positioned on EPD 200 such that the patient can place their thumbs or other fingers to contact the sensors, to provide an ECG recording (e.g. an additional ECG recording collected by system 10). For example, system 10 can perform diagnostic monitoring (e.g. ECG recording) on a predetermined schedule, but also allow for additional diagnostic monitoring (e.g. ECG recording) as determined by the patient (e.g. at any time). In some embodiments, the patient may choose to perform additional monitoring based on a physiologic condition, such as feeling dizzy, feeling faint, having palpitations, having shortness of breath, feeling tired, and the like. In some embodiments, the monitoring of the one or more patient parameters can be initiated by the patient. For example, the one or more patient parameters to be monitored (as initiated by the patient) can comprise at least an ECG, and the system can be configured to adjust the therapy provided (e.g. initiate stimulation energy delivery) based on detection of an arrhythmia via the monitored ECG. In some embodiments, the patient, clinician, and/or other user of system 10 can adjust the monitoring of one or more patient physiologic parameters, such as to establish a time-interval for monitoring of these parameters.

In some embodiments, functional element 299 of EPD 200, and/or another functional element of system 10, comprises one or more sensors that are configured to record EMG, EEG, and/or ECG, and system 10 is configured to analyze the recorded signals in order to perform a diagnosis and/or prognosis (“diagnosis” herein) of sleep apnea of the patient. EPD 200 can be configured to monitor one or more parameters related to the detection of sleep apnea selected from the group consisting of: movement, such as chest movement; snoring; body position; heart rate; O2 saturation; and combinations of these. In some embodiments, system 10 is configured to detect sleep apnea, such as when system 10 is further configured to transmit a signal (e.g. a haptic feedback signal) to the patient (e.g. a transmission that is delivered to stop or at least reduce the number of sleep apnea events that are occurring). In some embodiments, system 10 is configured to provide a sleep analysis. Analysis performed by system 10 (e.g. sleep apnea and/or other patient diagnosis such as a diagnosis of atrial fibrillation) can be accessible via an online portal (e.g. a patient portal hosted by server 600), and/or automated reports can be provided to the patient's managing physician.

EPD 200 can include processing unit 210 which can be configured to perform one or more functions of EPD 200. Processing unit 210 can include one or more algorithms, algorithm 215 shown. In some embodiments, processing unit 210 comprises a memory for storing instructions to perform algorithm 215. Processing unit 210 can be constructed and arranged to execute algorithm 215 and to thereby execute one or more functions of EPD 200. In some embodiments, processing unit 210 analyzes data (e.g. via algorithm 215) received from ID 100. For example, EPD 200 can receive data from ID 100, process (e.g. mathematically process) the information received via algorithm 215 (e.g. to determine if pacing should be performed, and to determine the parameters of stimulation energy to be delivered), and send information and/or power to ID 100 based on the processed information.

EPD 200 can include power module 240. Power module 240 can include one or more power-generating, power-harvesting, power-storing, and/or other power-supplying components configured to deliver energy to EPD 200, and/or to deliver power to ID 100 via wireless power transfer. Power module 140 can be configured to provide power to one or more components of EPD 200. In some embodiments, power module 240 comprises one or more batteries, capacitors, and/or other power-storing devices. Power module 240 can be constructed and arranged to “harvest” power from kinetic motion. In some embodiments, power module 140 comprises one or more piezo electric components configured to convert kinetic energy to electrical energy.

CD 300 can include one or more catheters and/or or one or more surgical tools for delivering ID 100 into the patient. Additionally, CD 300 can include one or more devices configured to diagnose and/or treat the patient, such as to perform a diagnosis and/or a treatment during a clinical procedure in which ID 100 is implanted into the patient. For example, CD 300 can comprise a cardiac mapping catheter which can be used to collect data (e.g. data to be processed by console 400) such as to map the cardiac electrical activity of the heart. Additionally or alternatively, CD 300 can comprise an ablation catheter which can be used to ablate tissue (e.g. cardiac tissue). In some embodiments, system 10 can include one or more clinician devices 300 that are constructed and arranged to enable the clinician to perform: a mapping procedure, a tissue treatment procedure (e.g. an ablation procedure or other tissue treatment procedure), and/or an ID 100 implantation procedure (e.g. for continued, post procedural treatment of the patient).

In some embodiments, CD 300 comprises electrode array 310 shown, which can comprise one or more arrays of electrodes that can be inserted into the patient. Electrode array 310 can comprise one or more electrodes 311. CD 300 can include user interface 350 shown. User interface 350 can include one or more user input and/or user output components, for example, one or more: displays, indicators (e.g. LEDs), speakers, buttons, levers, microphones, and/or other user interface devices. In some embodiments, user interface 350 comprises a handle (e.g. a catheter handle) including one or more controls, such as a steering control.

In some embodiments, CD 300 includes transceiver 320. Transceiver 320 can comprise an assembly configured to communicate (e.g. wirelessly communicate) with one or more components of system 10, for example, one or more implanted devices 100, one or more external patient devices 200, console 400, and/or other components of system 10. Transceiver 320 can comprise a receiving and/or transmitting interface, antenna 325. In some embodiments, CD 300 includes one or more functional elements, functional element 399 shown. Functional element 399 can include one or more sensors and/or transducers.

In some embodiments, system 10 includes a data storage and processing device, server 600. Server 600 can comprise an “off-site” server (e.g. outside of the operating room or other clinical site in which ID 100 is implanted), such as a server maintained by the manufacturer of system 10. Alternatively or additionally, server 600 can comprise a cloud-based server. Server 600 can include processing unit 610 shown, which can be configured to perform one or more functions of server 600. Processing unit 610 can include one or more algorithms, algorithm 615. In some embodiments, processing unit 610 includes a memory for storing instructions to perform algorithm 615. Processing unit 610 can be constructed and arranged to execute algorithm 615 and to thereby execute one or more functions of server 600. Server 600 can be configured to receive and store various forms of data, such as: patient, procedural, device, and/or other information, data 620. Data 620 can comprise data collected from multiple patients (e.g. multiple patients treated with system 10), such as data collected during and/or after clinical procedures where ID 100 was implanted into the patient. For example, data can be collected from ID 100, transmitted to EPD 200, and sent to server 600 for analysis. In some embodiments, one or more devices of system 10, such as EPD 200 and server 600, can communicate over a network, for example, a wide area network such as the Internet. In some embodiments, system 10 includes a virtual private network (VPN) through which various devices of system 10 transfer data.

Algorithm 615 can be configured to analyze data 620. For example, algorithm 615 can be configured to analyze data 620 collected from multiple patients to identify similarities and/or differences in treatment parameters and patient results. In some embodiments, algorithm 615 comprises a machine learning and/or other artificial intelligence algorithm (“AI algorithm” herein) that can be configured to identify patterns in the correlations between treatment parameters and results based on data collected from multiple patients. In some embodiments, algorithm 615 analyzes patterns to determine better treatment parameters for one or more patients to be treated using system 10. For example, algorithm 615 can identify one or more patterns in the data (e.g. one or more patterns associated with efficacy of the treatment being delivered to the patient) by analyzing data 620 collected from many patients (e.g. tens of thousands of patients). Algorithm 615 can be further configured to use these patterns to determine whether a patient (e.g. in the set of patients from which the data was collected and/or in a new patient) is receiving sub-optimal treatment (e.g. the parameters associated with pacing and/or other energy being delivered could be modified to improve efficacy). System 10 (e.g. via algorithm 615) can be configured to alert the clinician of a patient receiving sub-optimal treatment, and to recommend (e.g. via CD 300, such as the clinician's phone or computer) the parameters be adjusted. In some embodiments, the clinician may schedule an appointment to adjust the parameters (e.g. in person), or the parameters can be adjusted remotely, for example, when CD 300 is configured to adjust the parameters remotely via the network. Alternatively or additionally, server 600 can adjust the parameter automatically (e.g. via the network). In some embodiments, one or more parameters are automatically adjustable (e.g. within certain thresholds), while other parameters require clinician approval.

As described herein, system 10 can comprise one or more algorithms, such as algorithms 135, 215, 415 and/or 615 shown in FIG. 1. Various algorithms of system 10 can be referred to singly or collectively herein as algorithm 1005. In some embodiments, algorithm 1005 comprises a machine learning and/or other artificial intelligence algorithm (“AI algorithm” herein). Any algorithmic process described herein may be performed by any algorithm of system 10 (e.g. algorithms 135, 215, 415, and/or 615). The various processors and/or controllers of system 10 can each comprise memory configured to store instructions for performing the algorithms described herein. In some embodiments, algorithm 1005 is configured to analyze (e.g. compare, contrast, combine, and/or otherwise analyze) various therapeutic strategies (e.g. sets of diagnostic and/or therapeutic parameters) that can be provided by system 10 to treat a patient, such as to create a patient-specific strategy (e.g. a pacing strategy for that particular patient to be implemented by controller 130 of ID 100). In some embodiments, the patient-specific strategy is transmitted to server 600, such as to be analyzed by algorithm 615. In some embodiments, a set of patient-specific strategies created for multiple patients are analyzed by server 600, such as to improve algorithm 1005 for creating new patient-specific strategies. In some embodiments, the patient-specific strategies (e.g. sets of therapeutic and/or diagnostic parameters) are created using an AI algorithm.

System 10 can be configured to record electrical activity, such as cardiac electrical activity, and algorithm 1005 can be configured to analyze the recorded electrical activity. For example, electrical activity can be recorded via one or more electrodes 111 of electrode array 110. The recorded electrical activity can be transmitted, via transceiver 120, to EPD 200. Algorithm 215 of EPD 200 can be configured to analyze the received data, and to determine if stimulation is required to treat the patient. Algorithm 215 can determine a set of stimulation parameters to be delivered by ID 100 based on the received electrical data (e.g. based on a recorded pattern of conduction within the cardiac tissue). For example, algorithm 215 can determine the location and instances in time to deliver stimulation energy (e.g. via electrodes 111). Alternatively or additionally, the recorded electrical data can be transmitted to console 400 and/or server 600, such that algorithms 415 and/or 615 can analyze the data and determine stimulation parameters. The stimulation parameters determined by an algorithm 1005 can be transmitted back to implantable device 100, via transceiver 220, to ID 100. In some embodiments, the stimulation parameters prescribe stimulation pulses to be delivered as a sequence of pulses to be delivered simultaneously and/or asynchronously, and/or regularly and/or irregularly. The stimulation pulses can be delivered from one or more electrodes 111. In some embodiments, algorithm 135 is configured to process stimulation parameters received from EPD 200 and stimulate via electrodes 111 based on the processed parameters. Alternatively or additionally, ID 100 does not comprise an algorithm, and is configured to stimulate based on power and/or data received from EPD 200 (e.g. stimulation power is received by transceiver 120 and provided to an electrode 111 based on data received with the transmitted power).

In some embodiments, system 10 is configured to stimulate cardiac tissue by providing electrical stimulation that is below a perception threshold such that there is no perception of any pain and/or discomfort caused by the delivery of the electrical stimulation (e.g. the patient doesn't feel any pain or discomfort caused by the delivery of the electrical stimulation).

As described herein, system 10 can be configured to perform single and/or multisite pacing, such as to terminate AF and/or SVT of the patient. AF can be caused by a stretch-induced infiltration of fibrosis that is progressively and broadly distributed across the left atrium. Global, simultaneous mapping of AF has revealed patient-specific confined zones of conduction that are distributed primarily across three anatomical regions of the left atrium: (1) posterior wall; (2) anterior-roof; and (3) anterior-septum. In the early phase of AF, categorized as “paroxysmal”, the progression of fibrosis is more confined to the muscular sleeves surrounding the pulmonary veins and the posterior wall of the left atrium. As the disease of AF progresses into the “persistent” stage, fibrosis spreads beyond the posterior wall, predominantly emerging at patient-specific locations across the roof and septum, anteriorly.

The feasibility of low-voltage shocks and multisite pacing for terminating AF can be limited by: (1) the number, size, and/or distribution of electrodes placed about the left atrium; and (2) the pattern of stimulation energy delivered. The progressive nature of the disease requires matching the spatiotemporal characteristics of pacing with the patient-specific distribution of fibrosis.

In some embodiments, for example in the early, paroxysmal phase of AF, pacing can be delivered from multiple (e.g. 3 or 4) electrodes distributed within the Vein of Marshall and the adjacent coronary sinus. These locations are close to the lateral border of the posterior wall and the left pulmonary veins, where stimulation is required for effective interruption of fibrillatory conduction in the region of the left atrium that is relevant for paroxysmal AF.

In some embodiments, for example in the later persistent and long-standing phases of AF, pacing can be delivered from more electrodes (e.g. 5 or 6 electrodes) that are distributed epicardially on the posterior wall, the anterior roof, and/or superior septum. These locations are close to the critically, “confined zones” of conduction that maintain AF. Stimulation is required to be delivered near these zones for effective interruption of fibrillatory conduction in the regions of the left atrium that are relevant for persistent AF.

In some embodiments, one or more sensors (e.g. a functional element 199 comprising one or more sensors and/or one or more electrodes 111 configured as a sensor) of ID 100 (e.g. an ID 100 comprising one or more implantable devices) are positioned at one or more locations proximate heart tissue and are configured to produce signals from which a calculation of pressure within a chamber (e.g. pressure of the blood within the left atrium) and/or a calculation of a hydration level can be determined (e.g. by one or more of algorithms 1005), such as is described in reference to FIG. 1 herein. In some embodiments, the signals are recorded from (e.g. ID 100 and the associated sensors are implanted at) a location outside of the chamber of the heart for which the chamber pressure and/or a hydration level is determined (e.g. outside of the left atrium when left atrial pressure is determined). For example, one, two or more electrodes 111 of ID 100 (e.g. an ID 100 comprising one, two, or more implantable devices) can be configured to record signals to determine a chamber pressure and/or a hydration level, such as when an impedance measurement is performed to identify impedance characteristics of tissue surrounding a heart chamber (e.g. the left atrium) that can be correlated (e.g. by an algorithm 1005) to the chamber pressure (e.g. left atrial pressure) and/or a hydration level (e.g. changes in a hydration level). Alternatively or additionally, functional element 199 of ID 100 (e.g. an ID 100 comprising one, two, or more implantable devices) can comprise one, two, or more sensors configured to produce a signal (e.g. record a physiologic parameter) that can be used to determine the pressure of a heart chamber and a hydration level. In some embodiments, at least one, two, or more of these sensors are positioned within the chamber for which the chamber pressure and/or a hydration level is determined. In some embodiments, at least one, two, or more of these sensors are positioned on tissue that is proximate, but outside the chamber for which the chamber pressure is determined (e.g. on the epicardial wall), such as when no sensors are present within that chamber. Applicable sensors for producing a signal used by system 10 (e.g. an algorithm 1005) to determine a chamber pressure include but are not limited to: a pressure sensor; a strain gauge (e.g. to measure strain in tissue that can be correlated to the chamber pressure proximate the tissue on which the sensor is positioned); an accelerometer (e.g. to measure tissue motion that can be correlated to chamber pressure proximate the tissue on which the sensor is positioned); an ultrasound sensor and/or other acoustic sensor, such as a doppler ultrasound sensor (e.g. configured to measure one or more blood flow parameters which can be correlated to the chamber pressure); an optical sensor (e.g. configured to measure one or more tissue and/or blood properties which can be correlated to the chamber pressure); and combinations of these.

In some embodiments, ID 100 comprises one or more implantable devices that are configured to be implanted proximate the heart (e.g. to monitor and/or stimulate heart tissue as describe herein) and to function independently, for example without EPD 200, such as without need for power transfer from EPD 200 or any other separate device.

In some embodiments, ID 100 comprises two or more implantable devices, for example as described in reference to FIG. 25 herebelow. For example, ID 100 can comprise a first implantable device that is implanted proximate the epicardial surface, as described herein, and a second implantable device that is implanted away from the heart, for example in a subcutaneous pocket. In some embodiments, the second implantable device is configured to provide power to the first implantable device (e.g. to provide wireless power, as described in reference to FIG. 25 herebelow). In some embodiments, ID 100 (e.g. the second implantable device) comprises a device configured to defibrillate and/or pace the heart, such as when configured similar to an implantable cardioverter defibrillator (ICD) (e.g. configured to defibrillate the heart) and/or configured similar to a pacemaker (e.g. configured to pace the heart). Alternatively, ID 100 can comprise a single implantable device (e.g. a single device configured to be implanted on the epicardial surface) that is configured to perform, one, two, three or more functions selected from the group consisting of: defibrillate; pace; detect an arrhythmia (e.g. atrial fibrillation); treat an arrhythmia (e.g. atrial fibrillation); and combinations of these, as is described herein.

In some embodiments, system 10 is configured to detect and/or monitor for invasive hemodynamic congestion. For example, ID 100 can record electrical signals, such as signals correlating to left ventricular end diastolic pressure (LVEDP), and/or left atrial pressure (LAP). ID 100 can record signals periodically, such as for at least one minute per day. The recorded signals can be analyzed by system 10, such as by algorithm 1005 via ID 100, EPD 200, and/or server 600 (e.g. when the recorded signals are transmitted to server 600 for analysis by algorithm 1005). In some embodiments, algorithm 1005 can be configured to detect trends (e.g. undesirable trends) in LVEDP and/or LAP (e.g. trends detected in the recorded electrical signals over days and/or weeks). Trends detected by algorithm 1005 can be correlated to disease progression, and/or can be used to direct a therapy protocol (e.g. a guided diuretic therapy protocol). In some embodiments, system 10 can monitor LVEDP and/or LAP with at least 2 times, at least 4 times, and/or at least 8 times greater resolution than a current commercial device (e.g. a commercial wearable device) that is configured to monitor one or more of these patient parameters (e.g. as ID 100 would be less susceptible to the far-field and/or geometric averaging limitations of a wearable device).

In some embodiments, ID 100 comprises one or more redundant components. For example, ID 100 can comprise two or more of controllers 130 and/or two or more of transceivers 120. Redundant components can be configured to activate only if a primary component fails, such that ID 100 can continue to function if a primary component fails without need for removal and/or replacement of ID 100. For example, ID 100 can comprise two or more complete sensing and/or pacing circuits (e.g. controllers 130), such as two or more circuits (e.g. primary and backup circuits) that are each operably connected to electrode array 110, and/or each are operably connected to a unique electrode array 110 (e.g. when ID 100 comprises two or more electrode arrays 110). When the primary circuit fails, the backup circuit can be configured to activate.

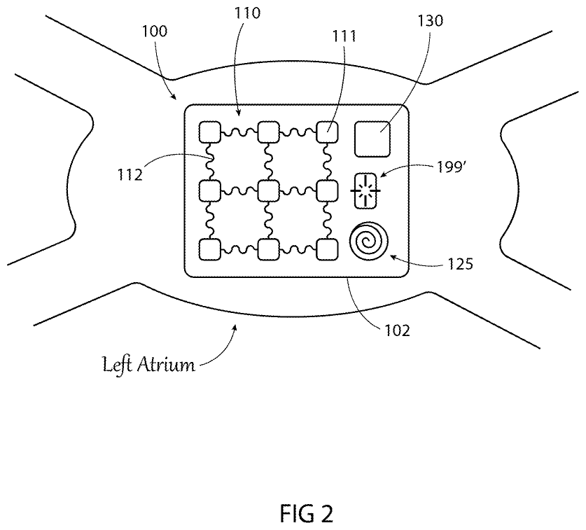

Referring now to FIG. 2, an anatomical view of an implantable device implanted proximate a left atrium is illustrated, consistent with the present inventive concepts. ID 100 of FIG. 2 can be of similar construction and arrangement to ID 100 described in reference to FIG. 1 herein and can include similar components (e.g. controller 130 as shown). ID 100 comprises electrode array 110 including multiple electrodes 111. Array 110 is positioned on and/or within substrate 102, for example, such that at least a surface portion of each electrode 111 is positioned to be in contact with the epicardial wall of the left atrium (e.g. when ID 100 is positioned on the epicardial surface of the atrium as shown). Electrodes 111 of array 110 are electrically connected to wires 112, as shown. Implantable device 110 can include controller 130 and/or antenna 125, also as described herein. In some embodiments, ID 100 comprises a functional element comprising an LED, LED 199′ shown.

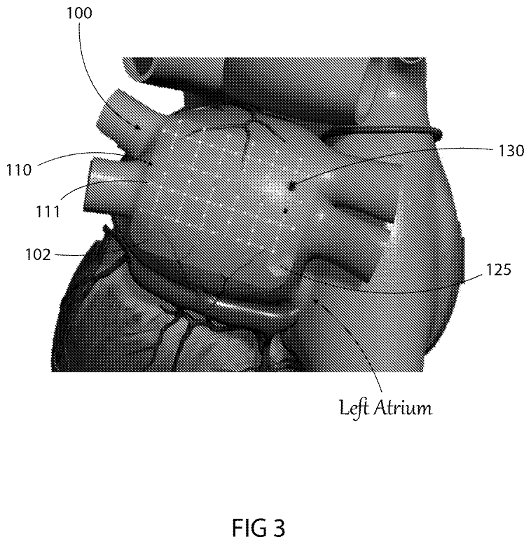

Referring now to FIG. 3, an artistic rendering of an implantable device positioned on the epicardial surface of a left atrium is illustrated, consistent with the present inventive concepts. ID 100 of FIG. 3 can be of similar construction and arrangement and can comprise similar components to ID 100 described in reference to FIG. 1 herein and can include similar components (e.g. controller 130 as shown).

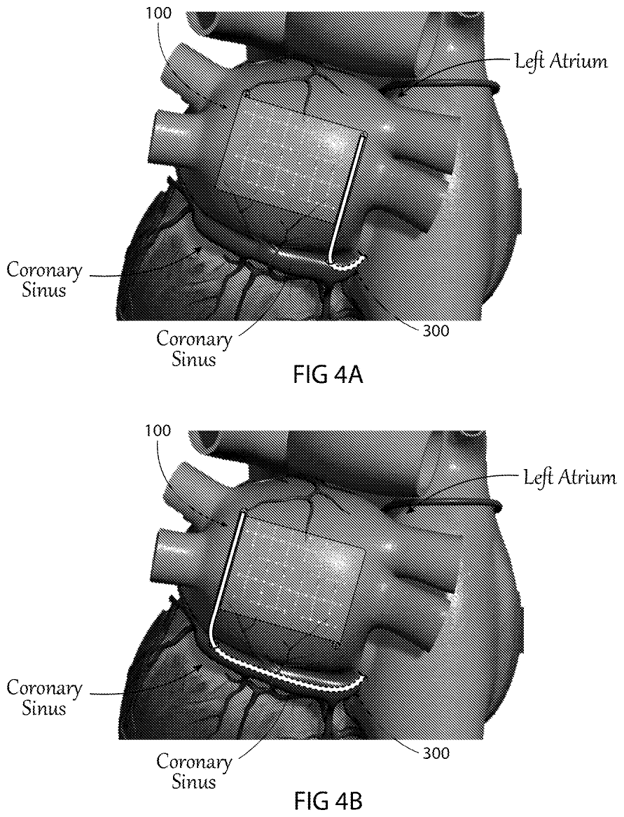

Referring now to FIGS. 4A and 4B, two artistic renderings of implantable devices being deployed from a clinician device onto the epicardial surface of a left atrium are illustrated, consistent with the present inventive concepts. Implantable devices 100 of FIGS. 4A and 4B can be of similar construction and arrangement, and can comprise similar components, to ID 100 described in reference to FIG. 1 herein and can include similar components. ID 100 of FIGS. 4A and 4B comprise an unfurlable design which can be constructed and arranged to be inserted into the patient percutaneously, advanced through the patient's vasculature, transvascularly advanced into the pericardial space, and deployed (e.g. unfurled) along the epicardial surface of the left atrium. CD 300 can comprise a delivery catheter, not shown but constructed and arranged to deliver ID 100 to the epicardial space, for example, via the coronary sinus. Alternatively or additionally, ID 100 comprising an unfurlable design, such as is shown in FIGS. 4A and 4B, can be implanted using non-transvascular methods, such as minimally invasive surgical methods. In some embodiments, the transvascular methods described herein can be used to deploy various configurations of ID 100, such as spiral configurations and/or multiple implantable devices 100 of a network of implantable devices 100.

In FIG. 4A, CD 300 is shown exiting a proximal portion of the coronary sinus (e.g. through the vein wall) into the epicardial space. In this embodiment, ID 100 can unfurl along the epicardial surface of the left atrium from right to left (as shown in the illustration). In FIG. 4B, CD 300 is shown exiting a distal portion of the coronary sinus (e.g. through the wall of the vein) into the epicardial space. ID 100 can be unfurled along the epicardial surface of the left atrium (e.g. from left to right as shown in the illustration).

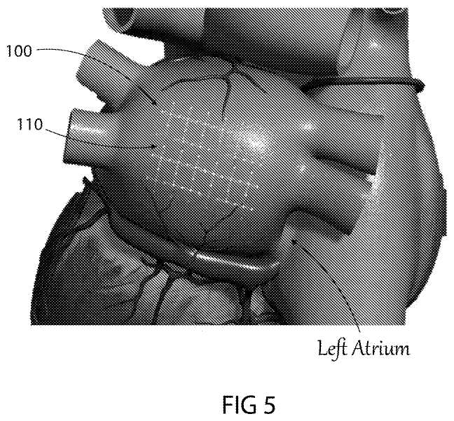

Referring now to FIG. 5, an artistic rendering of an electrode array positioned on the epicardial surface of a left atrium is illustrated, consistent with the present inventive concepts. ID 100 of FIG. 5 can be of similar construction and arrangement, and can comprise similar components, to ID 100 described in reference to FIG. 1 herein and can include similar components. In some embodiments, electrode array 110 of ID 100 is configured to be positioned directly onto tissue (e.g. the epicardial surface of the left atrium as shown in FIG. 5), for example, when ID 100 does not comprise a substrate (e.g. substrate 102 described herein). In some embodiments, CD 300 comprises a deposition device (e.g. a 3D printer) configured to deposit conductive and/or nonconductive material onto tissue, such as a device configured to “print” electrode array 110 directly onto tissue.

Referring now to FIGS. 6A and 6B, various configurations of wires connecting two electrodes of an electrode array are illustrated, consistent with the present inventive concepts. Wires 112 are shown connecting electrodes 111a and 111b. Electrodes 111a and 111b can comprise electrodes of an electrode array, such as electrode array 110 described herein. In FIG. 6A, wires 112 comprise a wavelike geometry (e.g. a sinusoidal geometry), configured to minimize Van der Waals, tensile, compressive, and/or other undesired forces that may be encountered (e.g. forces encountered by electrode array 110 when ID 100 undergoes contraction and/or elongation due to the motion of the heart). In FIG. 6B, wires 112 comprise a wavelike geometry with a greater frequency of oscillation than the wires of FIG. 6A. The frequency and/or amplitude of the “waves” of the geometry of the wires can be selected to minimize the undesired forces imparted on wires 112. In some embodiments, wires 112 are configured in a geometry that minimizes sharp angles (e.g. to minimize areas of high stress within wires 112).

Referring now to FIG. 6C, various configurations of a planar antenna are illustrated, consistent with the present inventive concepts. Antenna 125 can be of similar construction and arrangement to antenna 125 of FIG. 1 described herein. Antenna 125 can comprise a planar micro coil, such as a coil in the shape of a triangle, square, pentagon, hexagon, and/or a circle as shown. Antenna 125 can comprise one or more wires or other electrical conduits of ID 100, such as one or more wires configured to connect two or more components of ID 100 (e.g. a wire configured to connect an electrode 111 to controller 130 can also be configured as a part of antenna 125).

Referring now to FIG. 6D, an antenna configuration including an extension is illustrated, consistent with the present inventive concepts. Antenna 125 can be of similar construction and arrangement to antenna 125 of FIG. 1 described herein. Antenna 125 can comprise a first portion, antenna portion 1251, and a second portion, antenna portion 1252. Antenna portion 1252 can comprise a configuration dissimilar to the configuration of antenna portion 1251, such as a planar antenna positioned in a different plane (e.g. a plane perpendicular to the plane of antenna portion 1251), or a different shape, such as the coil shape shown. In some embodiments, antenna portion 1252 is configured to allow connections (e.g. transmissions) to an external device (e.g. EPD 200 not shown but described herein) when the external device is not properly positioned relative to first antenna portion 1251 (e.g. not perpendicular antenna portion 1251).

Referring now to FIG. 6E, a fractal antenna design and frequency chart are illustrated, consistent with the present inventive concepts. Antenna 125 can be of similar construction and arrangement to antenna 125 of FIG. 1 described herein. In some embodiments, antenna 125 comprises a fractal antenna, shown. A fractal antenna design can comprise multiple “self-similar” antenna sets, such as three sets of triangular-shaped antennas as shown, each set comprising triangles of a varying size. Alternatively or additionally, antennas with other self-similar geometries can be included, such as self-similar geometries comprising one or more lines, polygons, and/or curves. Each set of antennas are tuned to a particular “Self-Resonance Frequency” (SRF), shown.

In some embodiments, various pairs of electrodes of ID 100, not shown but described herein, are controlled via signals transmitted at particular frequencies to which the various antenna sets of antennae 125 are tuned (e.g. signals, such as signals comprising power and data transmitted from EPD 200, not shown). In some embodiments, a fractal antenna design enables energy harvesting over a broad range of frequencies. In some embodiments, a fractal antenna design enables EPD 200 to transmit power to ID 100 (via antenna 125) at multiple frequencies.

Referring additionally to FIG. 6F, an anatomic view including a magnification of an implantable device is illustrated, consistent with the present inventive concepts. ID 100 can include multiple implantable devices implanted proximate the heart, as shown. Each ID 100 can comprise an antenna 125, such as a fractal antenna, and two or more electrodes, such as electrodes 111a and 111b shown. EPD 200 can be configured to transmit power and/or data to implantable devices 100.

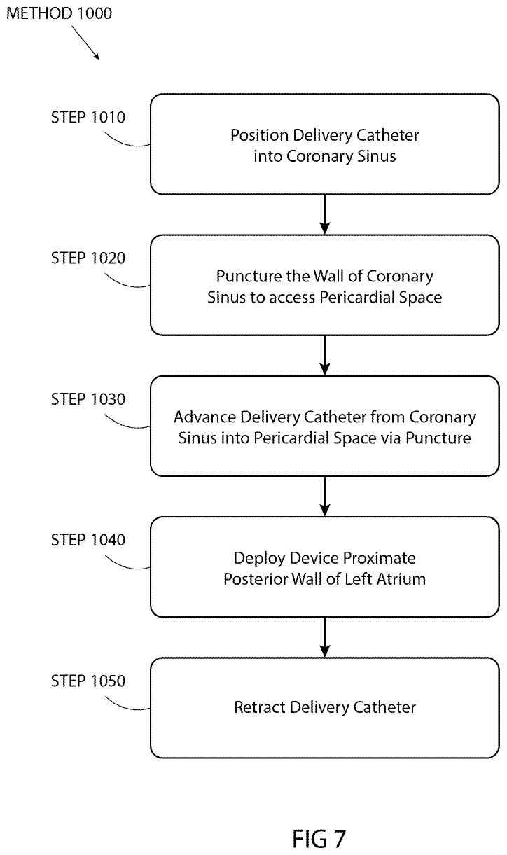

Referring now to FIG. 7, a method of delivering a device to the epicardial space is illustrated, consistent with the present inventive concepts. Method 1000 describes an interventional procedure in which ID 100 (e.g. ID 100 described herein) is advanced through the vasculature into the coronary sinus, and then transvascularly delivered to the epicardial space through the wall of the coronary sinus (or vessel wall proximate the coronary sinus). In Step 1010, CD 300 (e.g. a delivery catheter) is advanced through the vasculature of a patient and its distal portion is positioned within the coronary sinus. In Step 1020, the distal end of CD 300 punctures the wall of the coronary sinus, providing access to the epicardial space. CD 300 can comprise a needle configured to puncture the wall of the coronary sinus, for example, a needle no larger than 20 G for example, a 27 G needle. In some embodiments, device 300 punctures a tissue wall at a location near the proximal end of the coronary sinus, as illustrated in FIG. 4A. Alternatively, device 300 can puncture a tissue wall near the distal end of the coronary sinus, as illustrated in FIG. 4B. In some embodiments, ID 100 is configured to be deployed from either location, such that the clinician can decide intraoperatively which is the preferred anatomical location for deployment, for example, a preference determined based on the patient anatomy.

In Step 1030, the distal portion of CD 300 is advanced from the coronary sinus into the pericardial space. CD 300 can exit the coronary sinus toward the top of the posterior wall of the left atrium. In some embodiments, system 10 is constructed and arranged to insufflate the pericardial space, such as by introducing a gas into the space, for example, CO₂ gas.

In Step 1040, ID 100 is deployed from CD 300 and into the pericardial space along the posterior wall of the left atrium. In some embodiments, ID 100 comprises an unfurlable structure that is unfurled along the posterior wall. Alternatively or additionally, ID 100 can comprise another deployable form, such as the structure with a spiral geometry described in reference to FIG. 8 and otherwise herein. ID 100 can comprise a geometry biased in a deployed state that is maintained in an undeployed state (e.g. furled, non-spiraled, or other non-expanded state) while within CD 300. For example, once an ID 100 that is configured to expand (e.g. unfurl, transition to a spiral, and/or otherwise expand) is advanced from CD 300, the biased geometry can cause the device to automatically expand (e.g. unfurl, transition to a spiral, and/or otherwise expand) as it exits CD300.

In some embodiments, once deployed, ID 100 is secured to the tissue. For example, CD 300 can be constructed and arranged to suture, staple, and/or otherwise using a securing element to secure ID 100 to the tissue. Alternatively, ID 100 can be attached to tissue via an adhesive, such as adhesive 70 described in reference to FIG. 1 herein. In some embodiments, CD 300 is constructed and arranged to deliver adhesive 70 between ID 100 and the tissue. Alternatively or additionally, ID 100 can be pre-treated (e.g. include) with adhesive 70, such as when adhesive 70 comprises an activatable adhesive. For example, adhesive 70 can comprise an adhesive configured to be activated by ultraviolet light and/or other activation mechanism. In some embodiments, CD 300 can be configured to emit ultraviolet light to activate adhesive 70.

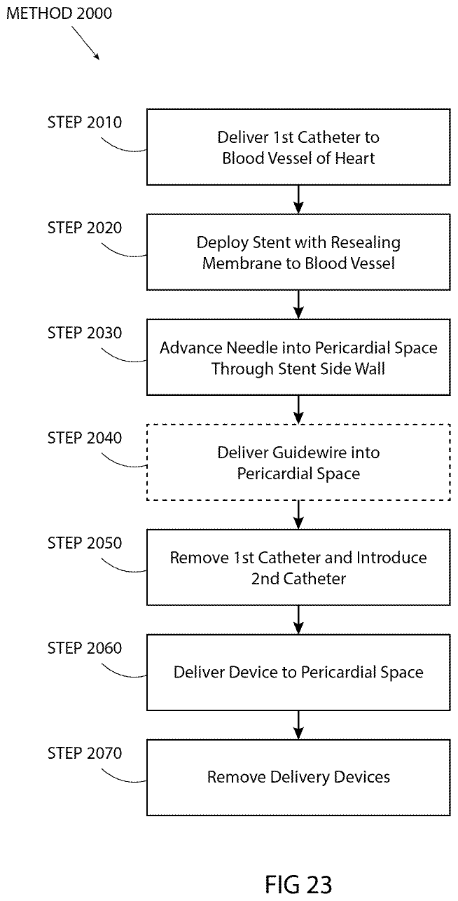

In Step 1050, CD 300 is retracted from the patient, leaving ID 100 implanted in the patient. In some embodiments, the pericardial space is deflated, desufflated, and/or equilibrated prior to and/or after the removal of CD 300. In some embodiments, system 10 includes a sealing device configured to prevent blood flow from the heart (e.g. from the coronary sinus) into the pericardial space, for example after CD 300 is removed, such as is described in reference to FIG. 23 herein. In some embodiments, CD 300 is configured to aspirate blood from the pericardial space, such as blood from the heart that entered the pericardial space (e.g. when CD 300 punctured the wall of the coronary sinus, and/or while CD 300 was positioned through the wall of the coronary sinus into the pericardial space).

Referring now to FIG. 8, a perspective view of an implantable device comprising a spiral geometry is illustrated, consistent with the present inventive concepts. ID 100 of FIG. 8 can be of similar construction and arrangement to ID 100 described in reference to FIG. 1 herein, and it can include similar components (e.g. controller 130 as shown). ID 100 can comprise a spiral geometry, as shown. Substrate 102 can comprise an elongate tubular structure, shaft 1021, such as a multi-layer tubular structure comprising one or more tubular inner layers (e.g. a nickel-titanium tubular frame) surrounded by one or more protective outer tubular layers (e.g. a polymer coating). In some embodiments, electrodes 111 of electrode array 110 comprise ring shaped electrodes that surround a portion of shaft 1021 (e.g. as shown). In some embodiments, wires 112 extend through shaft 1021 (e.g. through a lumen of shaft 1021), for example, wires 112 can extend from controller 130 to each electrode 111.

ID 100 can be constructed and arranged to be deployed from CD 300 (as shown). CD 300 can comprise a catheter including a lumen that can slidingly receive ID 100. In some embodiments, ID 100 is resiliently biased in the spiral geometry shown. When positioned within CD 300, ID 100 can assume a linear geometry (while constrained within the lumen of CD 300) and can be configured to automatically transition to the spiral geometry shown as it is advanced from CD 300 into the pericardial space.

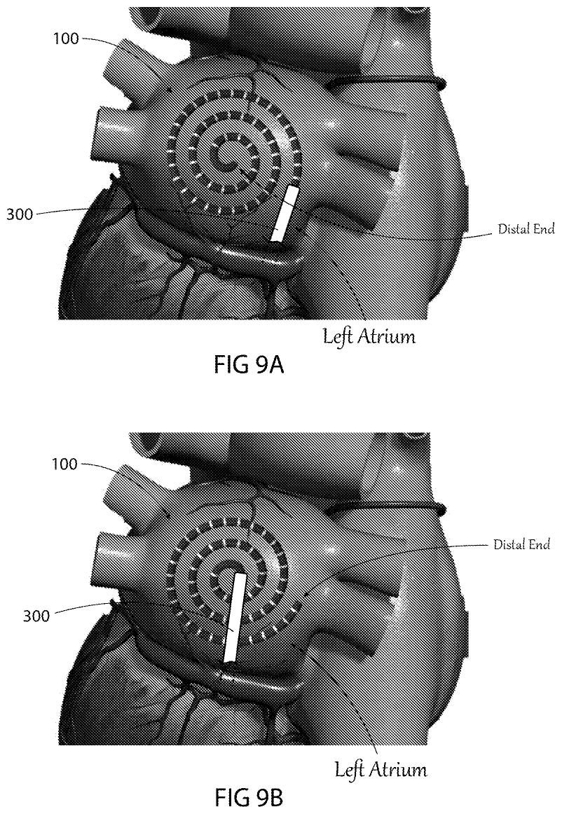

Referring now to FIGS. 9A and 9B, artistic renderings of implantable devices comprising spiral geometries each positioned on the epicardial surface of a left atrium are illustrated, consistent with the present inventive concepts. Implantable devices 100 of FIGS. 9A and 9B can be of similar construction and arrangement, and can comprise similar components, to ID 100 described in reference to FIG. 1 herein. In some embodiments, ID 100 is constructed and arranged such that when deployed the distal end of ID 100 is positioned at the relative center of the spiral geometry of ID 100, as shown in FIG. 9A. Alternatively or additionally, ID 100 can be constructed and arranged such that when deployed the distal end of ID 100 is positioned along the outer diameter of the spiral geometry, as shown in FIG. 9B. In FIGS. 9A and 9B, IDs 100 are shown extending from the distal end of clinician device 300, such as a clinician device comprising a catheter extending from the coronary sinus, or a surgical tool configured to access the pericardial space, as described herein.

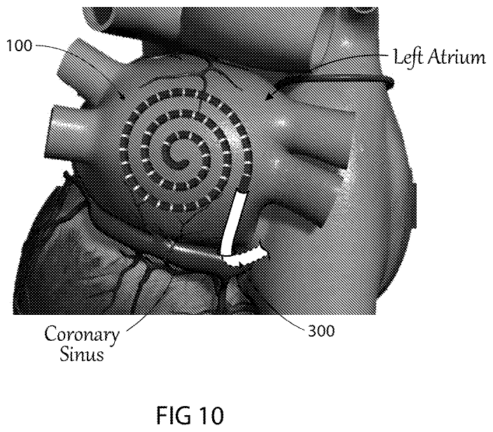

Referring now to FIG. 10, an artistic rendering of an implantable device comprising a spiral geometry being deployed from a clinician device onto the epicardial surface of a left atrium is illustrated, consistent with the present inventive concepts. ID 100 of FIG. 10 can be of similar construction and arrangement, and can comprise similar components, to ID 100 described in reference to FIG. 1 herein. ID 100 of FIG. 10 can comprise a resiliently biased spiral geometry, for example, as described in reference to FIG. 8 herein. ID 100 can be constructed and arranged to be implanted into the patient in an interventional procedure, such as when ID 100 deploys into a spiral geometry while being advanced from a catheter-based CD 300. CD 300 can comprise a delivery catheter constructed and arranged to deliver ID 100 to the epicardial space, for example, via the coronary sinus. For example, CD 300 can transvascularly exit the proximal portion of the coronary sinus, through the vein wall, into the epicardial space (as shown).

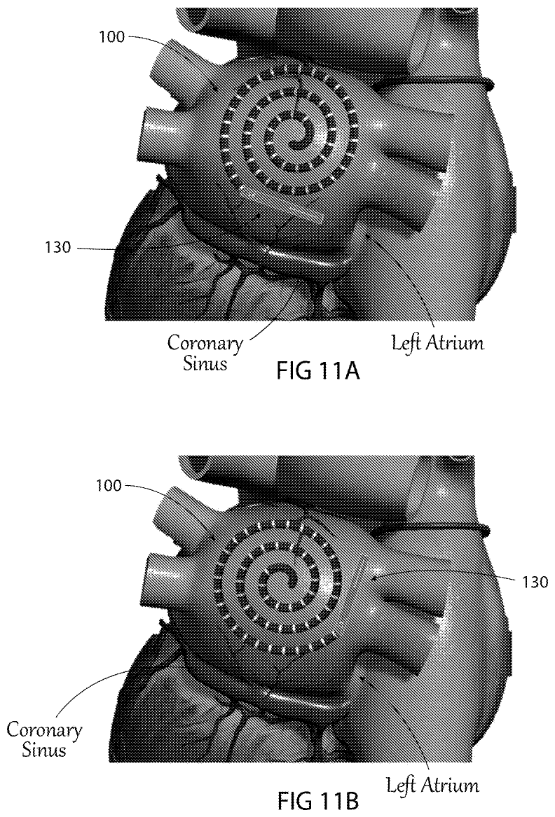

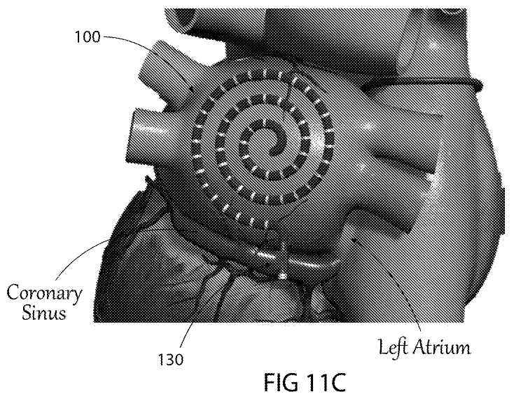

Referring now to FIGS. 11A-11C, various artistic renderings of implantable devices each positioned on the epicardial surface of a left atrium and each comprising an electronic assembly are illustrated, consistent with the present inventive concepts. Implantable devices 100 of FIGS. 11A-11C can be of similar construction and arrangement and can comprise similar components (e.g. controller 130 as shown), to ID 100 described in reference to FIG. 1 herein. Each embodiment of ID 100 of FIGS. 11A-C illustrate a possible implantation configuration of controller 130 of ID 100. For example, as shown in FIG. 11A, controller 130 can comprise a linear geometry which can be implanted parallel to the lumen of the coronary sinus, as shown. Alternatively, as shown in FIG. 11B, controller 130 can comprise a linear geometry which can be implanted orthogonally to the lumen of the coronary sinus, as shown. In some embodiments, as shown in FIG. 11C, controller 130 comprises a housing constructed and arranged to be implanted proximate the coronary sinus, for example, proximate the location from which CD 300 exited the coronary sinus during implantation of ID 100.

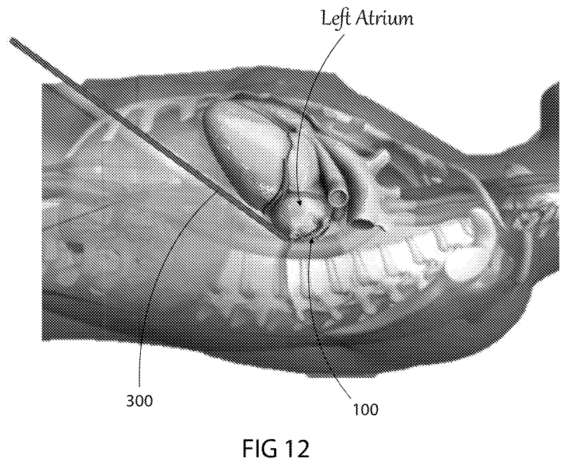

Referring now to FIG. 12, a partially transparent anatomic view of an implantable device being implanted along an epicardial surface is illustrated, consistent with the present inventive concepts. Implantable device 100 and/or clinician device 300 of FIG. 12 can be of similar construction and arrangement and can comprise similar components to devices 100 and/or 300 described in reference to FIG. 1 herein. Clinician device 300 can comprise an elongate device configured to extend through the skin of the patient (e.g. via a subxiphoid incision) and into the pericardial space. Clinician device 300 can comprise a lumen through which ID 100 is deployed along an epicardial surface (e.g. the epicardial surface of the left atrium). In some embodiments, clinician device 300 comprises one, two, or more devices constructed and arranged to allow for deployment of ID 100 along the epicardial surface in a minimally invasive surgery, such as devices including a trocar, a guide wire, and/or an elongate device (e.g. a push rod) configured to position ID 100 at a desired implant location. In some embodiments, clinician device 300 comprises a steerable portion, such as a robotically steerable portion constructed and arranged to maneuver in 3D space within a patient to access a deployment site for ID 100.

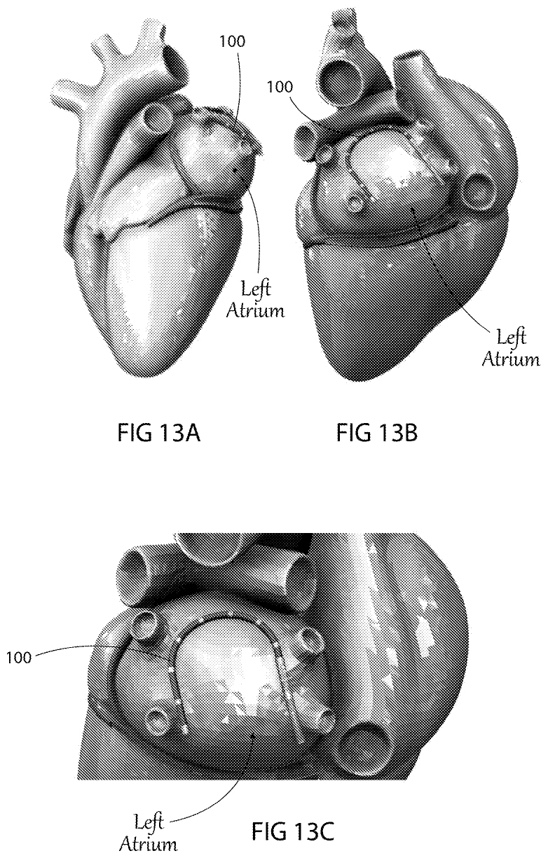

Referring now to FIGS. 13A-C, various anatomic views of the heart showing an implantable device positioned on the epicardial surface are illustrated, consistent with the present inventive concepts. Implantable device 100 of FIG. 13 can be of similar construction and arrangement and can comprise similar components to ID 100 described in reference to FIG. 1 herein. In some embodiments, ID 100 is positioned along the epicardial surface of the left atrium, between the pulmonary veins, as shown.

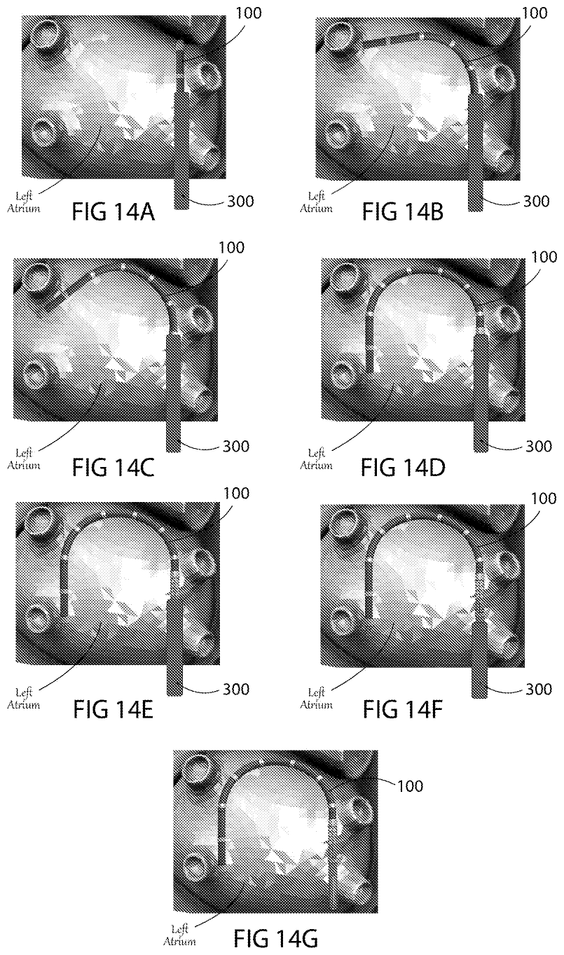

Referring now to FIGS. 14A-G, sequential anatomic images showing the deployment of an implantable device along the epicardial surface are illustrated, consistent with the present inventive concepts. Implantable device 100 and/or clinician device 300 of FIGS. 14A-G can be of similar construction and arrangement and can comprise similar components to devices 100 and/or 300 described in reference to FIG. 1 herein. In some embodiments, clinician device 300 comprises an intravascular device, such as an intravascular device which has been delivered through a vessel wall (e.g. the wall of the coronary sinus), such as to be positioned along the epicardial surface of the left atrium as shown. Alternatively or additionally, clinician device 300 can comprise a surgical device, such as is described in reference to FIG. 12 herein, that has been positioned along the epicardial surface, as shown. In FIG. 14A, ID 100 has been partially extended from the distal end of clinician device 300. In FIG. 14B, ID 100 has been extended further from clinician device 300 and steered along the perimeter of the epicardial surface of the left atrium as shown. In FIGS. 14C-14F, ID 100 is further extended and steered to take the shape shown in FIG. 14G. In FIG. 14G, clinician device 300 has been removed (e.g. retracted from the patient, thereby leaving ID 100 implanted along the epicardial surface).

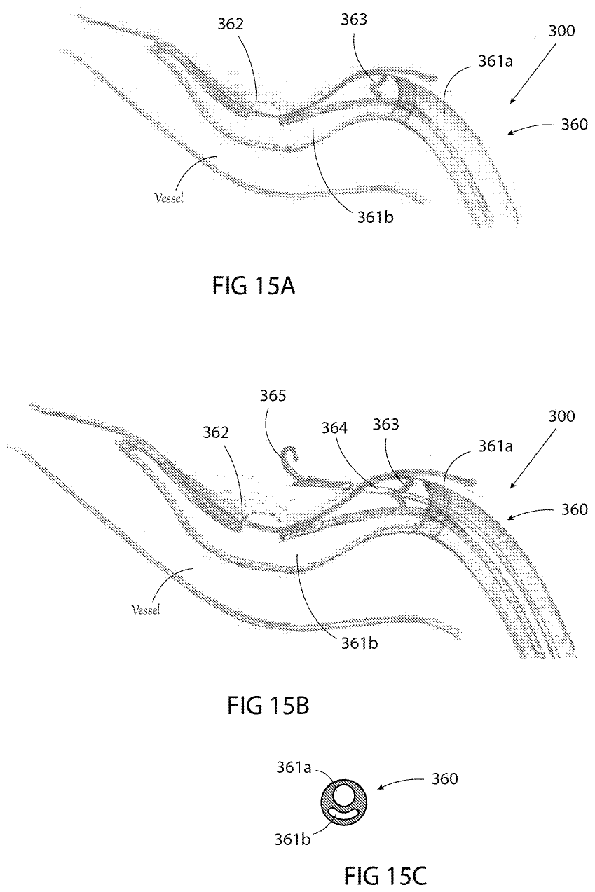

Referring now to FIGS. 15A-C, two sectional views of a device positioned within a vessel, and a cross sectional view of the device are illustrated, consistent with the present inventive concepts. Clinician device 300 of FIGS. 15A-C can be of similar construction and arrangement and can comprise similar components to device 300 described in reference to FIG. 1 herein. Clinician device 300 can comprise an elongate device, catheter 360, which can be configured to transvascularly deliver ID 100 into the epicardial space. Catheter 360 can comprise one, two, or more lumens 361, such as lumens 361a and 361b shown. Lumen 361b can comprise a vacuum lumen (e.g. can be operably attached on its proximal end to a source of vacuum), and can comprise one, two, or more openings, such as port 362 proximate its distal end. Port 362 can be constructed and arranged to removably adhere to the vessel wall (e.g. a vein or artery wall), for example, when vacuum is applied to lumen 361b, such as when tissue of the vessel wall is drawn into port 362 via the applied vacuum. Clinician device 300 can further comprise one, two, or more devices configured to puncture the wall of the vessel and access the epicardial space, such as needle 364 and/or guidewire 365. Needle 364 can slidingly receive guidewire 365. Lumen 361a can slidingly receive one or more devices (e.g. one at a time or multiple devices simultaneously), for example, needle 364, guidewire 365, and/or ID 100. In some embodiments, lumen 361a comprises a seal, seal 363, which can be configured to allow a device to exit lumen 361a while limiting ingress of blood or other contaminants into lumen 361a (e.g. while providing a sealing function).

In some embodiments, after catheter 360 has been positioned proximate a pericardial access site (e.g. within the coronary sinus), vacuum is applied to lumen 361b to adhere port 362 to the vessel wall to secure catheter 360. After catheter 360 is secured, needle 364 can be advanced from lumen 361a (e.g. through seal 363) and through the vessel wall into the epicardial space. Guidewire 365 can also be advanced into the epicardial space (e.g. via a lumen of needle 364) to maintain transvascular access into the space. In some embodiments, after access has been achieved, catheter 360 is removed from the patient (e.g. with guidewire 365 being left in the patient), and ID 100 can be inserted using guidewire 365 to guide the access to the epicardial space. Alternatively, ID 100 can be advanced through lumen 361a, or another lumen of catheter 360, and into the epicardial space.

Referring now to FIG. 16, an implantable device is illustrated, consistent with the present inventive concepts. Implantable device 100 of FIG. 16 can be of similar construction and arrangement and can comprise similar components to ID 100 described in reference to FIG. 1 herein. In some embodiments, ID 100 comprises a network of two or more implantable devices 100 configured to communicate with each other, such as to wirelessly communicate with each other. Alternatively or additionally, the network of implantable devices 100 can be configured to communicate (e.g. wirelessly communicate) with external patient device 200, not shown, but described herein.

Referring additionally to FIGS. 16A and 16B, various anatomic views showing potential placement locations of multiple implantable devices are illustrated, consistent with the present inventive concepts. Each implantable device 100 of the network of implantable devices 100 can be positioned on an endocardial surface location (e.g. within a heart chamber), within a vessel of the heart, and/or along an epicardial surface location. In FIG. 16A, a network of implantable devices 100a-e are shown positioned about the epicardial surface of the left atrium of the heart. In FIG. 16B, various possible placement locations 1-11 are illustrated about the left atrium of the heart.

Referring additionally to FIG. 16C, an anatomic view indicating pacing locations is illustrated, consistent with the present inventive concepts. In some embodiments, system 10 comprises between 12 and 22 electrodes 111, such as 16 shown. Multiple electrodes (or multiple implantable devices 100 each comprising at least one electrode 111) enables multisite pacing from various locations on and/or within the heart. In some embodiments, sets of one or more electrodes are positioned proximate the left atrium, such as sets positioned at locations 1-4 shown: 1, the proximal coronary sinus; 2, the distal Vein of Marshal; 3, the high septal area; and 4, the mid-posterior area. These placement examples of the 16 electrodes 111 shown enable numerous unipolar pacing combinations from various electrodes 111, and four bipolar pacing combinations.

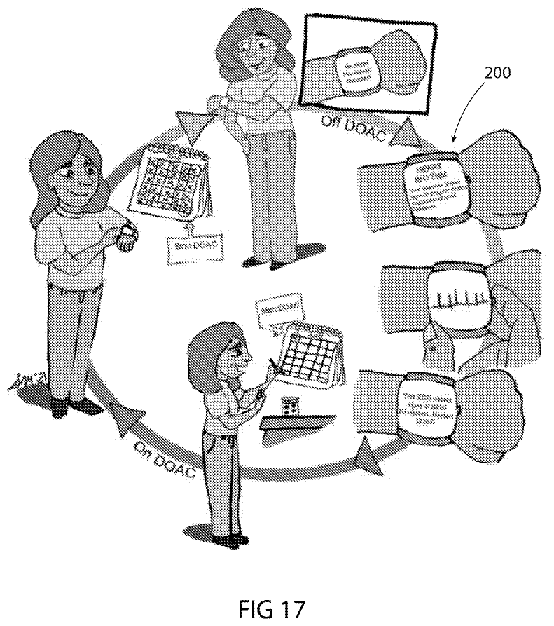

Referring now to FIG. 17, a method of managing medication is illustrated, consistent with the present inventive concepts. In some embodiments, system 10 monitors a patient parameter that is managed with medication, or it monitors side effects that are mitigated with medication. For example, patients with atrial fibrillation may take anticoagulants, such as direct oral anticoagulants (DOAC), to lower the likelihood of clot formation that is associated with the patient's fibrillation. Additionally or alternatively, the patient may take a diuretic medication for heart failure. System 10 can be configured to detect heart failure by monitoring left atrial pressure for di-composition, and/or by monitoring ejection fraction. System 10 can be configured to monitor for signs of an undesired patient condition, and if the condition is detected, system 10 can alert the user (e.g. via external patient device 200, such as an external device comprising a smart watch) to take a prescribed medication (e.g. to restart a prescribed medication). Additionally or alternatively, system 10 can be configured to inform the patient of a lack of an undesired condition (e.g. a healthy state), such as to cause the cessation of taking a medication.

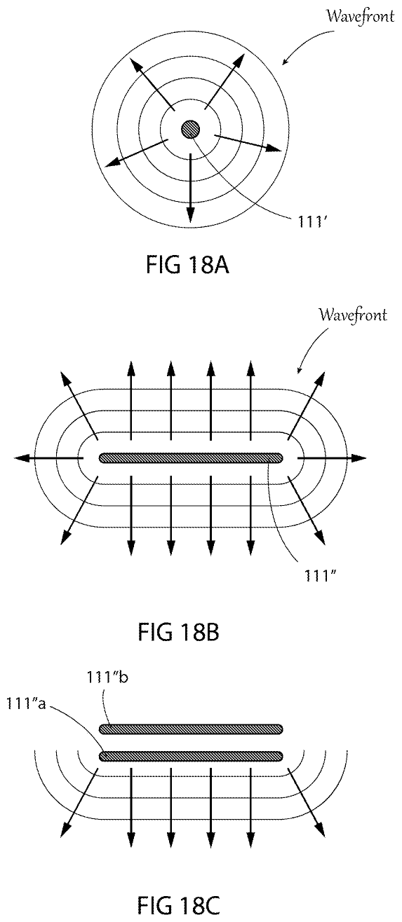

Referring now to FIGS. 18A and 18B, various electrode shapes are illustrated, consistent with the present inventive concepts. FIG. 18A shows a circular electrode 111′ (e.g. a “single point” electrode), and a representation of the electrical wavefronts generated in tissue when a stimulation pulse is delivered from electrode 111′. FIG. 18B shows an elongated linear electrode 111″, and a representation of the electrical wavefronts generated in tissue when a stimulation pulse is delivered from electrode 111″. An elongate electrode 111″ can be used to generate a partially planar wave as shown, which can provide a greater effectiveness and/or efficiency of stimulation than a single point electrode can provide. For example, a planar wave can affect a larger and broader area of tissue per unit of time than a single point electrode.

Referring additionally to FIG. 18C, an arrangement of multiple elongate electrodes is illustrated, consistent with the present inventive concepts. Two elongate electrodes 111″a and 111″b are shown positioned in a parallel arrangement, along with a representation of the electrical wavefronts generated when a bipolar stimulation pulse is delivered between electrodes 111″a and 111″b. A bipolar pulse delivered between electrodes 111″a and 111″b induces a planar wave propagating away from the cathodal electrode in all directions and causes anodal block proximate the anodal electrode, as shown. For example, the wave can propagate in all directions away from the first electrode 111″a, while simultaneously approaching the zone of block surrounding the second electrode and 111″b, ultimately, rapidly activating the zone immediately following the end of the simulation pulse.

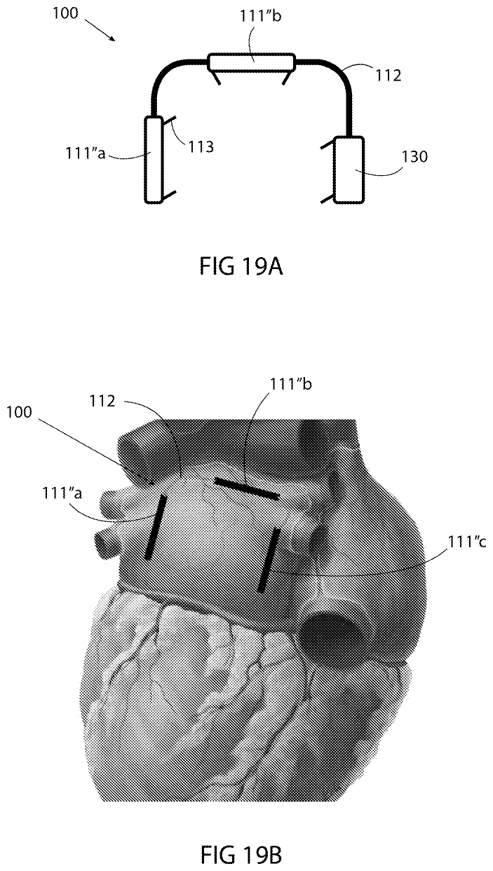

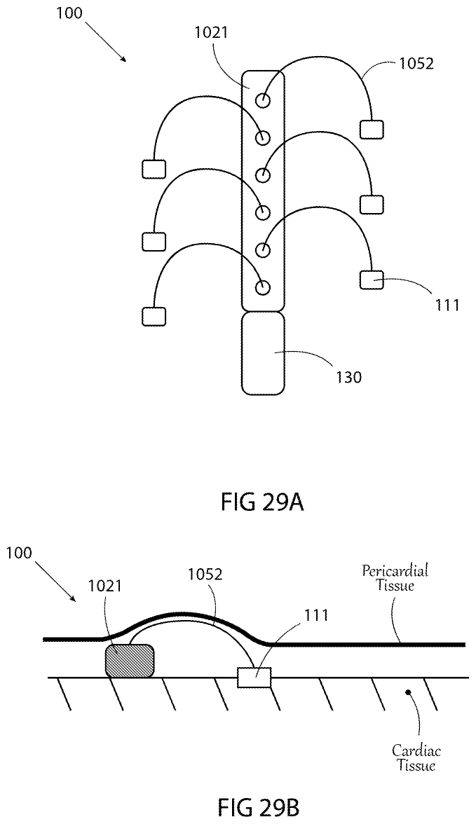

Referring now to FIGS. 19A and 19B, a schematic view of an implantable device and an anatomic view of the heart showing an implantable device positioned on the epicardial surface are illustrated, respectively, consistent with the present inventive concepts. Implantable device 100 of FIGS. 19A and 19B can be of similar construction and arrangement and can comprise similar components to ID 100 described in reference to FIG. 1 herein. In some embodiments, ID 100 comprises an elongate device biased in a curved geometry, such as the “U” shaped geometry shown. For example, ID 100 can comprise a flexible device configured to be straightened during delivery through a catheter (e.g. clinician device 300), and to transition into a biased curved geometry when deployed from the catheter. In some embodiments, ID 100 comprises one, two, or more elongate electrodes, such as electrodes 111″a and 111″b shown. Elongate electrodes 111″ can be similar to electrodes 111″ described in reference to FIG. 18B herein. Elongate electrodes 111″ can be connected by wires 112 (e.g. one or more wires within a shaft surrounding the wires). Wires 112 can comprise insulated wires such that electrical energy is only delivered to tissue from elongate electrodes 111″. In some embodiments, ID 100 comprises one, two, or more tissue anchors configured to anchor the device to tissue. For example, electrodes 111″ and/or controller 130 can each comprise one or more anchoring elements, barbs 113 shown. Barbs 113 can be configured to engage tissue (e.g. to puncture and/or pinch) to affix one or more portions of ID 100 to the tissue (e.g. to the epicardial surface). In some embodiments, barbs 113 are inward facing, as shown relative to electrode 111″b. Alternatively, barbs 113 can face in a single direction, as shown relative to electrode 111″a and controller 130, such that barbs 113 engage tissue by pulling against the direction of the barbs. In some embodiments, ID 100 comprises one or more projections that can be configured to push against the pericardial sac to anchor ID 100, for example as described in reference to FIGS. 28A through 29B and otherwise herein.

As shown in FIG. 19B, ID 100 can comprise multiple elongate electrodes, electrodes 111″a-c shown, configured to be placed about the epicardial surface of the left atrium as shown. In some embodiments, one or more electrodes 111 can be electrically connected (e.g. shorted together) to act as an elongate electrode 111″. In some embodiments, an elongate electrode 111″ can comprise two, three, or more electrodes 111 that are configured to operate independently and/or as a set to form an elongate electrode 111″.

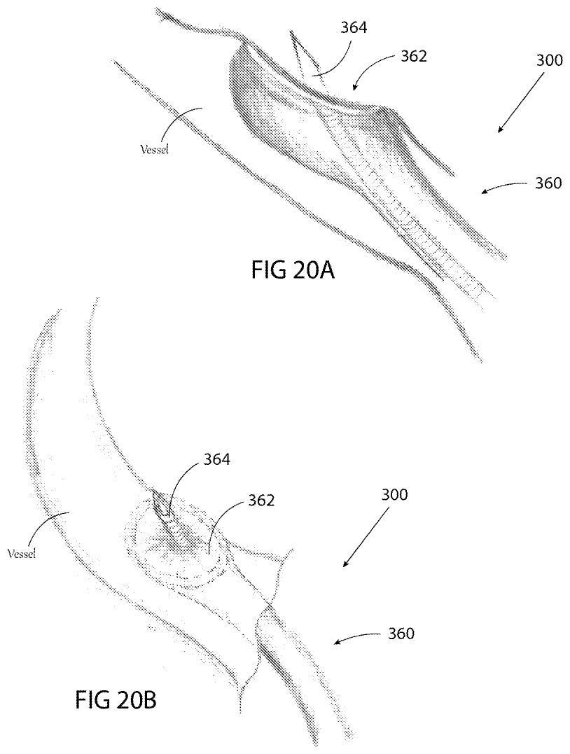

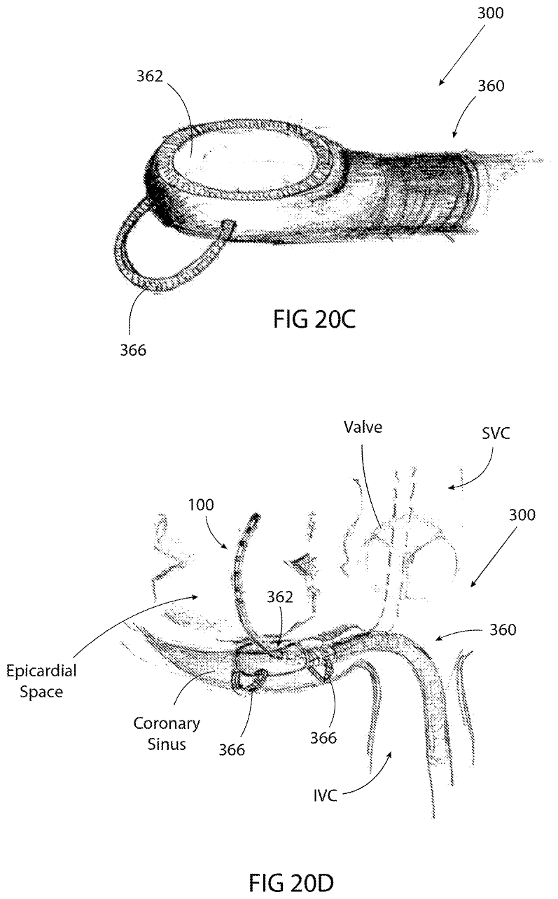

Referring now to FIGS. 20A-20D, various perspective views of a clinician device are illustrated, consistent with the present inventive concepts. In FIGS. 20A, B and D, the device has been inserted into a patient, with the tissue shown partially transparent for illustrative clarity. Clinician device 300 of FIGS. 20A-D can be of similar construction and arrangement and can comprise similar components to device 300 described in reference to FIG. 1 herein. Clinician device 300 can comprise an elongate device, catheter 360, which can be configured to transvascularly deliver ID 100 into the epicardial space. Catheter 360 can comprise port 362, which can be operably attached to a source of vacuum (e.g. via a lumen of catheter 360). Port 362 can be constructed and arranged to removably adhere to the vessel wall (e.g. a vein or artery wall), for example, when vacuum is applied to port 362, such as when tissue of the vessel wall is at least partially drawn into port 362 via the applied vacuum. Clinician device 300 can further comprise one, two, or more devices configured to puncture the wall of the vessel and access the epicardial space, such as needle 364.

In some embodiments, catheter 360 comprises one, two, or more tissue fixation elements, anchors 366 shown in FIGS. 20C and 20D. Anchors 366 can extend away from the opening of port 362, such as to oppose the vessel wall opposite the opening of port 362, forcing the port toward the opposing vessel wall. In some embodiments, catheter 360 is configured to access the coronary sinus via the inferior vena cava (IVC), and/or the superior vena cava (SVC) (e.g. through a valve as shown). As shown in FIG. 20D, one or more devices, such as ID 100, can be transvascularly advanced through port 362 into the epicardial space (e.g. after a puncture through the vessel wall is created with needle 364).

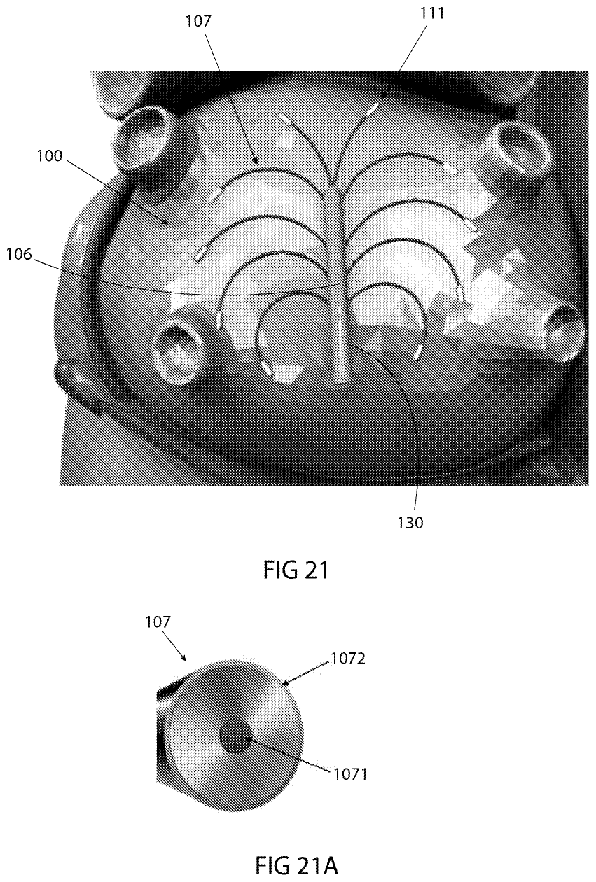

Referring now to FIGS. 21 and 21A, an anatomic view of an implantable device positioned on the epicardial surface and a cross section view of a portion of the device are illustrated, consistent with the present inventive concepts. ID 100 can comprise similar components to device 100 described in reference to FIG. 1 herein. ID 100 can comprise a deployable array including one or more filaments, deployable arms 107. Arms 107 can each be attached (e.g. attached at one end as shown) to a central structure, hub 106. Arms 107 can be configured to be collapsed toward hub 106 such that device 100 can be positioned within a delivery sheath (e.g. within clinician device 300, not shown, but comprising a delivery sheath). Each arm 107 can comprise one or more electrodes 111 of electrode array 110, such as one electrode 111 positioned on a distal portion of each arm 107, as shown. In some embodiments, controller 130 is positioned within hub 106 and operably (e.g. electrically) connects to each electrode 111, such as via a conductive portion of each arm 107. For example, each arm 107 can comprise a conductive core, core 1071, surrounded by an outer layer, cladding 1072, such as is shown in FIG. 21A. In some embodiments cladding 1072 comprises a nickel titanium alloy cladding. In some embodiments, arms 107 (e.g. arms 107 comprising a shape memory alloy such as nickel titanium) can comprise a set shape, such as a heat set shape. In some embodiments, cladding 1072 comprises an outer layer (e.g. a coating), such as a Parylene coating or a Tetrafluoroethylene coating configured to electrically insulate cladding 1072.

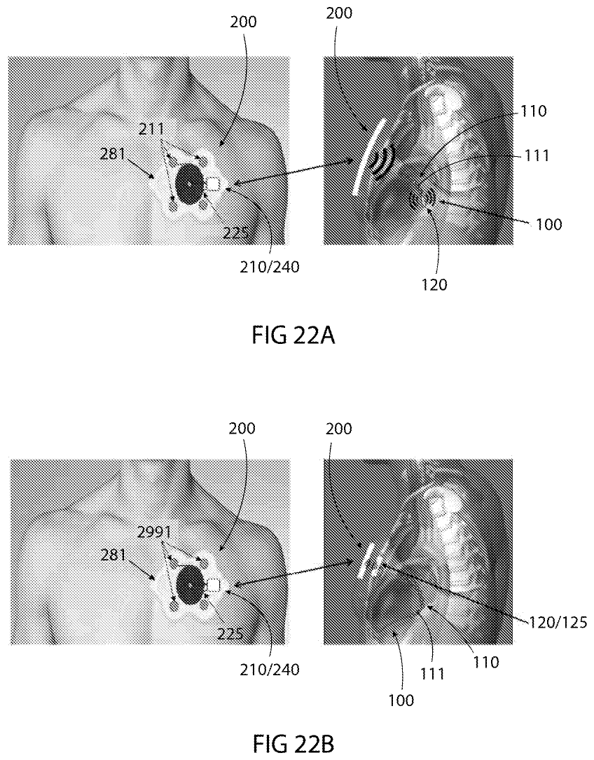

Referring now to FIGS. 22A and 22B, pairs of front anatomic and partially transparent side anatomic views of a patient wearing an external device are illustrated, consistent with the present inventive concepts. External patient device 200 and implantable device 100 can be of similar construction and arrangement to devices 200 and 100 of FIG. 1 described herein. In some embodiments, attachment assembly 280 of EPD 200 comprises an adhesive membrane, patch 281. Patch 281 can be configured to temporarily adhere to the skin of the patient, such as to the patient's chest proximate the heart, as shown. In some embodiments, EPD 200 comprises one or more functional elements, such as one or more electrodes 2991 configured to record patient electrical information. In some embodiments, EPD 200 comprises a disposable patch 281, that is removably attached to one or more reusable (e.g. rechargeable and reusable) components of EPD 200, such as antenna 225, processing unit 210, and/or power module 240. In some embodiments, one patch 281 comprises one or more disposable components of EPD 200 configured to operably attach to processing unit 210, such as electrodes 2991 and/or antenna 225, that are operably attached to processing unit 210 when processing unit 210 is attached to patch 281.

In some embodiments, ID 100, including electrode array 110 of electrodes 111 and transceiver 120 is positioned proximate the heart, such as on the epicardial surface as shown in FIG. 22A. Alternatively or additionally, at least a portion of ID 100, such as at least a portion of transceiver 120 (e.g. antenna 125) can be implanted sub dermally proximate the position of EPD 200 (as shown in FIG. 22B), such as to decrease the wireless power transfer distance. In these embodiments, transceiver 120 can be operably attached to other components of ID 100 (e.g. electrodes 111) via an electrical conduit that is tunneled through the tissue.

Referring now to FIG. 23, a method of implanting a device in the pericardial space is illustrated, consistent with the present inventive concepts. Method 2000 describes an interventional procedure in which ID 100 (e.g. ID 100 described herein) is advanced through the vasculature, and then transvascularly delivered to the epicardial space through the wall of a vessel. In Step 2010, a first delivery catheter (e.g. a first delivery catheter of CD 300) is advanced through the vasculature of a patient and its distal portion is positioned within a target vessel. In Step 2020, a stent comprising a resealing membrane is deployed into the target vessel. In Step 2030, a needle is advanced into the pericardial space through the resealable membrane and the target vessel wall.

In optional Step 2040, a guidewire is advanced into the pericardial space. In Step 2050, the first delivery catheter is removed, and a second delivery catheter is introduced into the pericardial space (e.g. a second delivery catheter loaded with ID 100 is introduced over the guidewire). Alternatively or additionally, ID 100 is loaded into the first delivery catheter (e.g. advanced to the distal end of the first delivery catheter from a proximal portion of the catheter), and the first delivery catheter is advanced into the pericardial space.

In Step 2060, ID 100 is delivered to the pericardial space. In Step 2070, all delivery devices are removed from the patient (e.g. retracted from the pericardial space through the wall of the target vessel and the resealing membrane of the stent). The resealing membrane of the stent delivered in Step 2020 can be configured to reseal after delivery devices are removed, preventing or at least limiting bleeding from the target vessel.

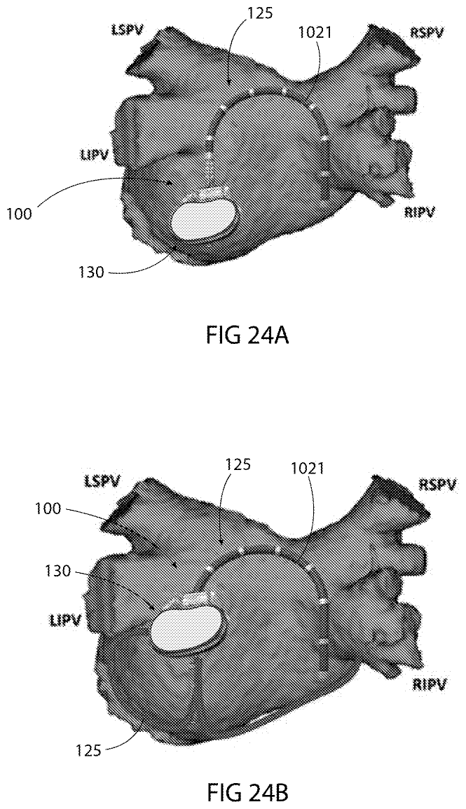

Referring now to FIGS. 24A and 24B, anatomic views of an implantable device positioned on the epicardial surface of a left atrium are illustrated, consistent with the present inventive concepts. Implantable device 100 can be similar to implantable device 100 of FIG. 1 described herein. In some embodiments, the entirety of ID 100 is positioned on the epicardial surface of the heart within the pericardial space (e.g. ID 100 does not include an implantable pulse generator (IPG), configured to be positioned away from the heart, outside of the pericardial space). In some embodiments, ID 100 and EPD 200 (not shown but described herein) are configured for bidirectional transmission of commands, recorded electrical signals (e.g. EGMs), and/or event recordings. In some embodiments, ID 100 comprises a single controller 130, such as a single ASIC chip.

As shown in FIG. 24B, antenna 125 of ID 100 can comprise a relatively large loop configuration (e.g. large relative to ID 100), such as when antenna 125 is configured to be looped around anatomic structures of the heart, such as the AV-groove and/or regions of the right or left atria. In some embodiments, a portion of antenna 125 can be positioned within and/or along a portion of shaft 1021, shown extending from controller 130. Antenna 125 can be positioned to optimize the geometry and/or orientation of the antenna to maximize the efficiency of power and/or data transfer. Additionally or alternatively, antenna 125 can comprise a fractal geometry, such as is described herein. In some embodiments, the majority of power can be transferred across the broader, coarser dimensions of the geometry of antenna 125 (such as by using a fixed frequency and/or a narrow band of frequencies). Data can be transferred across the narrower, finer dimensions of the geometry of antenna 125, using a broad band of frequencies. In some embodiments, power conversion efficiency can be optimized using a fractal switch-capacitor architecture.

Referring now to FIG. 25, a schematic anatomic view of an implantable system is illustrated, consistent with the present inventive concepts. System 10 can comprise a first implantable device, ID 100a, including an implantable housing (e.g. an IPG) operably attached via a lead to an electrode 111a implanted on an endocardial surface in the right atrium. ID 100a can also comprise antenna 125a implanted on an endocardial surface in the right atrium. In some embodiments, a single lead (e.g. a lead comprising two or more conductors) attaches both electrode 111a and antenna 125a to ID 100a (as shown). Alternatively or additionally, each of electrode 111a and antenna 125a are attached to ID 100a via an individual lead (e.g. such as when ID 100a comprises multiple attachment points for operably connecting to various leads). System 10 can also include a second implantable device, ID 100b, comprising electrode array 110a including at least one electrode 111a, implanted on the epicardial wall of the left atrium. ID 100b can include antenna 125b, positioned proximate antenna 125a. ID 100a can be configured to transmit power and/or data to ID 100b via antennas 125a, b such as is described herein. IDs 100a, b can include controllers 130a,b, shown, that are configured to exchange power and/or data (e.g. via antennas 125a,b) as described herein. In some embodiments, ID 100a includes a rechargeable battery, such as a battery that is configured to be wirelessly recharged by an external device (e.g. EPD 200 not shown but described herein).

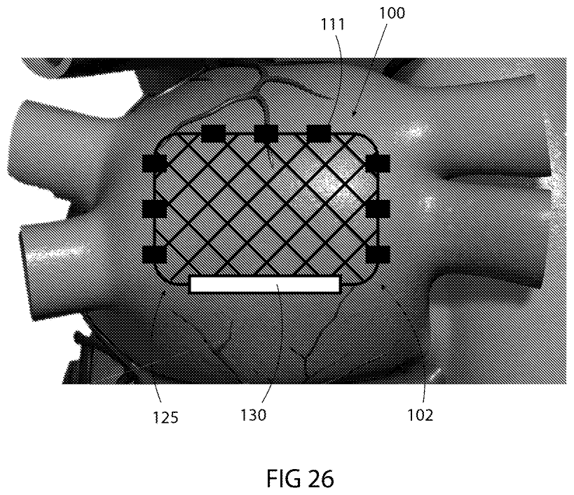

Referring now to FIG. 26, a schematic view of an implantable device positioned on the epicardial surface of a left atrium is illustrated, consistent with the present inventive concepts. Implantable device 100 can be similar to implantable device 100 of FIG. 1 described herein. Implantable device 100 can comprise substrate 102, with electrodes 111 positioned along the perimeter of substrate 102 as shown. Controller 130 can be positioned along an edge of substrate 102 (such that substrate 102 extends laterally from controller 130, as shown). In some embodiments, substrate 102 comprises a mesh-like configuration configured to promote ingrowth of tissue to anchor ID 100 (e.g. ingrowth of cardiac tissue to anchor ID 100 to the epicardial surface).

In some embodiments, substrate 102 includes a mesh of conductors (e.g. one or more insulated conductive wires woven into a mesh configuration) configured as antenna 125.

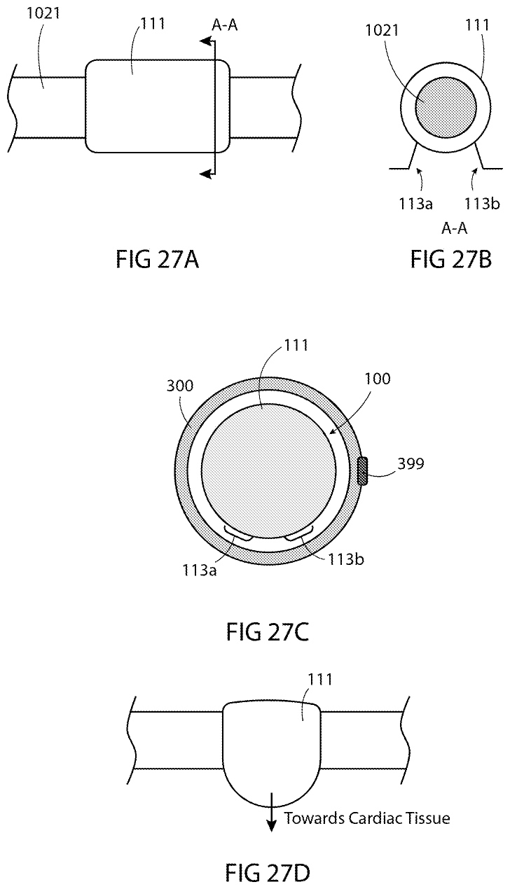

Referring now to FIGS. 27A-C, a side view and two end views of a portion of a device including an electrode with tissue anchors are illustrated, consistent with the present inventive concepts. FIG. 27A shows a top view of an electrode 111 surrounding a portion of shaft 1021. FIG. 27B shows a cross sectional view of electrode 111 along section A-A of FIG. 27A. Electrode 111 surrounds shaft 1021. Electrode 111 can comprise one or more anchoring elements, barbs 113a,b shown. Barbs 113 can comprise pivotable arms including tissue penetrating tips that engage tissue. In some embodiments, barbs 113 are attached to electrode 111 with a hinge mechanism, such as a biased mechanism biased to position barbs 113 as shown. Alternatively or additionally, barbs 113 can comprise a shape memory material, such as a nickel titanium alloy. Barbs 113 can be configured to transition (e.g. bend and/or pivot) to a collapsed configuration as shown in FIG. 27C, when ID 100 is positioned within a lumen of clinician device 300 (e.g. prior to deployment of ID 100). After exiting (e.g. being deployed from) the lumen of clinician device 300, barbs 113 can be configured to actuate, transitioning from the collapsed configuration of FIG. 27C to the deployed configuration of FIG. 27B, such as to secure electrode 111 to the cardiac tissue.

Clinician device 300 can include functional element 399, comprising a sensing electrode, positioned near the distal end of the lumen of clinician device 300. Signals recorded by functional element 399 can be analyzed (e.g. by algorithm 415 of console 400, not shown but described herein) to determine the location of the distal end of clinician device 300. The distal end of clinician device 300 can be manipulated (e.g. manipulated robotically, semi-robotically, and/or manually by a clinician using pull wires or other catheter steering mechanisms of clinician device 300) in a closed loop fashion such that electrodes 111 can be deployed in a known location (e.g. a predetermined location decided in a planning procedure prior to the implantation of ID 100). In some embodiments, stimulation energy is delivered and/or signals are recorded by functional element 399 to measure phrenic nerve stimulation thresholds pre, peri, and/or post deployment of an electrode 111 of ID 100.

Referring additionally to FIG. 27D, a side view of an electrode is illustrated, consistent with the present inventive concepts. Electrode 111 can comprise an asymmetric profile, with a protrusion extending towards (e.g. to protrude into) the cardiac tissue when implanted.

Referring now to FIGS. 28A-D, side and perspective views of an implantable device including a tissue anchor are illustrated, consistent with the present inventive concepts. ID 100 can be of similar construction and arrangement to ID 100 of FIG. 1 described herein. ID 100 can include shaft 1021, with electrodes 111 surrounding the shaft. In some embodiments, ID 100 comprises a commercially available mapping catheter, operably attachable to a signal generator (e.g. controller 130 and/or an energy delivery module 430 of console 400, not shown but described herein). ID 100 can comprise an insulative sleeve, sheath 1041, configured to electrically isolate electrodes 111 from tissue not intended to receive stimulation energy, such as the phrenic nerve and/or other electrically active thoracic structures. Sheath 1041 can comprise openings 1042, oriented towards the cardiac tissue when ID 100 is positioned on the epicardial surface of the heart, such that electrodes 111 are in electrical contact with the cardiac tissue, and are electrically isolated from the pericardial tissue.

ID 100 can comprise one or more anchoring elements 105, comprising filament projections, wings 1051. Wings 1051 can comprise a biased geometry, deflecting away from openings 1042, such that wings 1051 push against the pericardial sac when ID 100 is implanted on the epicardial surface, as shown in FIG. 28C. Wings 1051 can also be configured to prevent or at least limit axial rotation of shaft 1021. FIG. 28D illustrates various configurations of wings 1051.

Referring now to FIGS. 29A and 29B, a side view and a sectional anatomic view of an implantable device with anchoring elements are illustrated, respectively, consistent with the present inventive concepts. ID 100 can be of similar construction and arrangement to ID 100 described in reference to FIG. 1 herein. ID 100 can comprise shaft 1021 extending from controller 130. Each electrode 111 can be operably connected to shaft 1021 (e.g. electrically connected to controller 130 via shaft 1021) via a filament configured to anchor ID 100, anchor arm 1052. Arms 1052 can comprise resiliently biased arms configured to oppose pericardial tissue when ID 100 is implanted on the epicardial surface, as shown in FIG. 29B. In some embodiments, arms 1052 comprise a conductive core surrounded by an insulating cladding, such as a cladding comprising an elastic and/or shape memory material (e.g. a nickel titanium alloy).

Referring now to FIGS. 30A and 30B, side views of various anchoring elements are illustrated, consistent with the present inventive concepts. Electrode 111 shown in FIG. 30A can be of similar construction and arrangement to electrode 111 described in reference to FIG. 1 and otherwise herein. Electrode 111 can comprise one or more anchoring elements, barb 113, as shown. In some embodiments, electrode 111 comprises a conductive portion, and a non-conductive portion, as shown, such as when electrode 111 comprises coating 104. ID 100 of FIG. 30B can be of similar construction and arrangement to ID 100 described in reference to FIG. 1 herein. Electrodes 111 can be positioned on shaft 1021, and an anchoring element, wing 1051 can extend from shaft 1021, as shown. In some embodiments, wing 1051 comprises a filament with one or more anchoring elements, barbs 1053, that engage tissue (e.g. pericardial tissue) to anchor ID 100 when implanted to prevent or at least limit unwanted movement of ID 100.

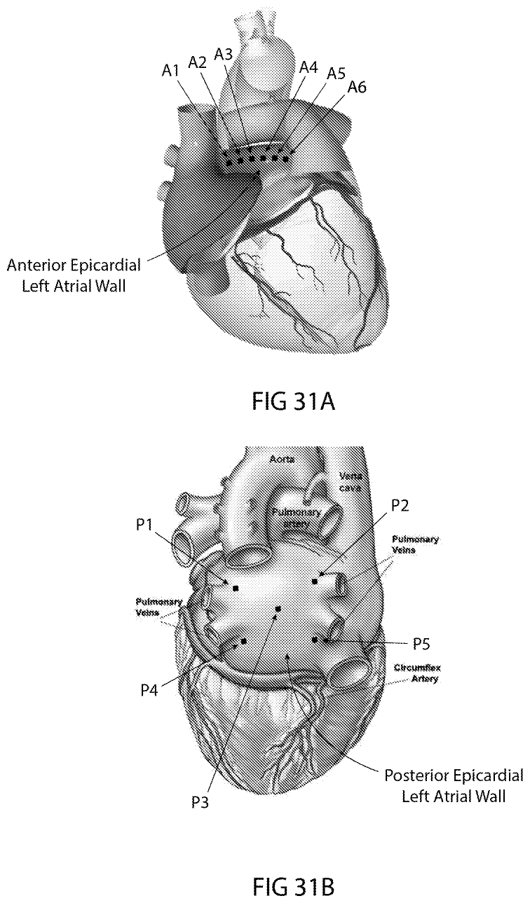

Referring now to FIGS. 31A and 31B, anterior and posterior views, respectively, of a heart are illustrated, indicating potential electrode implantation locations, consistent with the present inventive concepts. FIG. 31A shows six potential electrode locations for placement during an implantation procedure, locations A1-A6 shown, these locations positioned on the anterior epicardial wall of the left atrium. FIG. 31B shows five potential electrode locations for placement during an implantation procedure, locations P1-P5 shown, these locations positioned on the posterior epicardial wall of the left atrium. System 10 can include one or more IDs 100 that are configured to be implanted such that at least one electrode 111 is positioned at one or more of locations A1-A6 and/or P1-P5. For example, electrode placement combinations can be selected from the group consisting of: A1-A6; A2 and A5; A1, A3, and A5; A2, A4, and A6; P1-P5; P1, P2, and P3; P1, P3, and P5; P1 and P2; P1 and P3; P1 and P4; P1 and P5; P2 and P3; P2 and P4; P2 and P5; combinations of groups of one or more A locations and one or more P locations; and combinations of these.

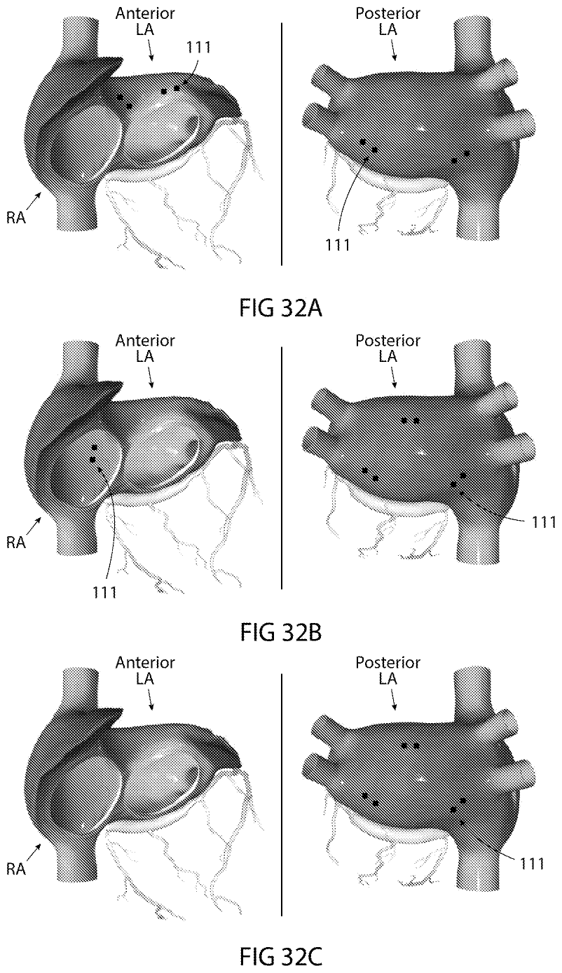

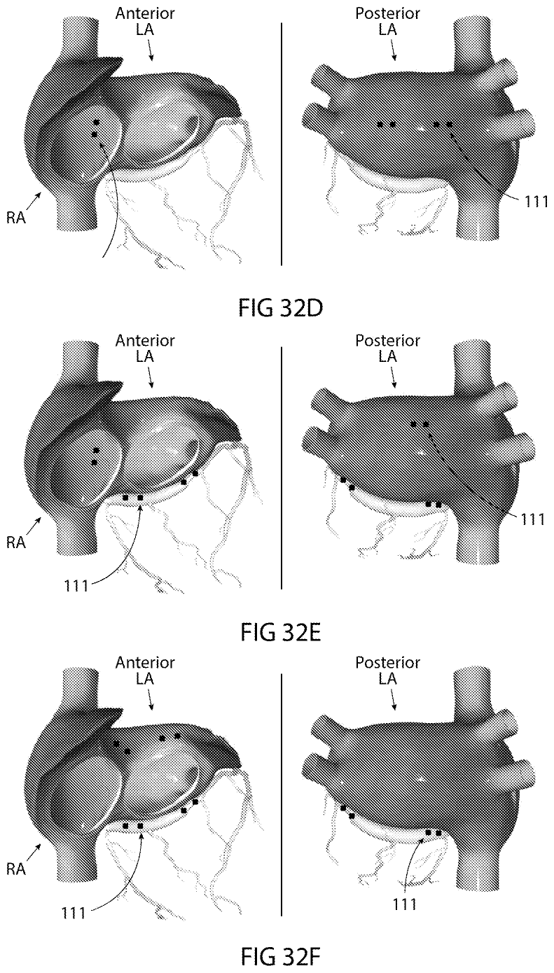

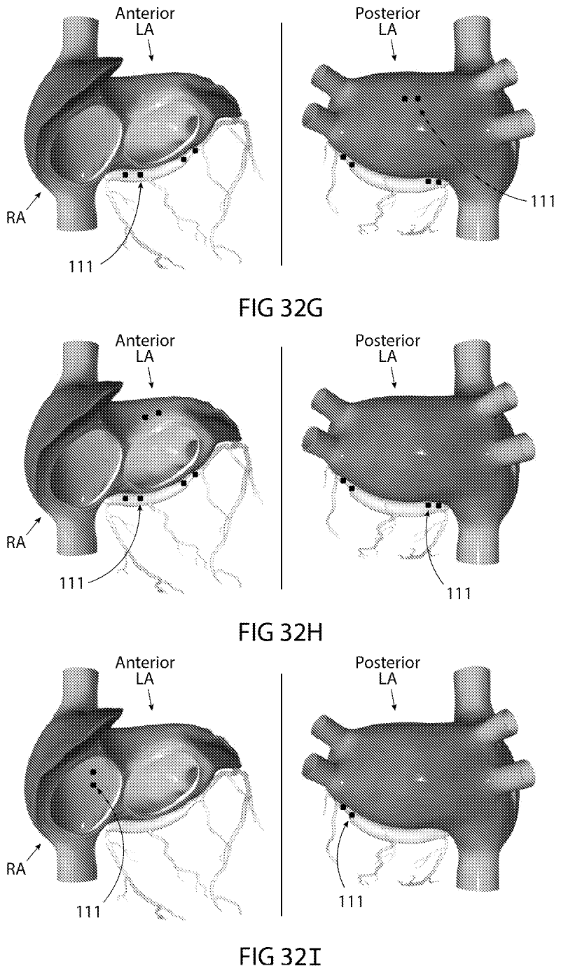

Referring now to FIGS. 32A-I, pairs of anterior and posterior anatomic views of the left atrium showing potential electrode implantation location configurations are illustrated, consistent with the present inventive concepts. Each of FIGS. 32A-I show a set of a minimal combination of electrodes 111 (e.g. pairs of electrodes 111) that can be implanted in particular anatomical locations such that the left atrium can be adequately and globally captured (e.g. both temporally and spatially). In some embodiments, electrodes 111 are positioned in direct contact with atrial tissue (e.g. one, two, or more electrodes 111 on the endocardial surface of an atrium, the epicardial surface of an atrium, or both). One or more electrodes 111 can be positioned (e.g. implanted proximate) any “region” of either atria, including regions located in anterior, posterior, lateral, septal, and/or inferior portions of each atria. For each of these regions, one or more electrodes 111 can be placed in any sub-portion of each region, including superior, inferior, lateral, and/or medial sub-portions of each region.

FIG. 32A shows multiple pairs of electrodes 111, the pairs positioned on (e.g. implanted proximate) the anterior epicardial wall of the left atrium in the septal and lateral regions; and on the posterior epicardial wall of the left atrium in the infero-lateral and infero-septal regions.

FIG. 32B shows multiple pairs of electrodes 111, the pairs positioned high on the endocardial surface of the right atrial septum, and on the posterior epicardial wall of the left atrium high on the mid-posterior region, as well as on the infero-lateral and infero-septal regions.

FIG. 32C shows multiple pairs of electrodes 111, the pairs positioned on the posterior epicardial wall of the left atrium high on the mid-posterior region, as well as on the infero-lateral and infero-septal regions.

FIG. 32D shows multiple pairs of electrodes 111, the pairs positioned high on the endocardial surface of the right atrial septum, and on the posterior epicardial wall in the low posterior region.

FIG. 32E shows multiple pairs of electrodes 111, the pairs positioned high on the endocardial surface of the right atrial septum, and on the posterior epicardial wall of the left atrium high on the posterior region. Pairs of electrodes 111 are also shown positioned endovascularly, in the lateral and septal portions of the coronary sinus.

FIG. 32F shows multiple pairs of electrodes 111, the pairs positioned on the epicardial wall of the left atrium on the septal and lateral anterior regions and positioned endovascularly in the lateral and septal portions of the coronary sinus.

FIG. 32G shows multiple pairs of electrodes 111, the pairs positioned on the posterior epicardial wall of the left atrium high on the posterior region and positioned endovascularly in the lateral and septal portions of the coronary sinus.

FIG. 32H shows multiple pairs of electrodes 111, the pairs positioned on the epicardial wall of the left atrium on the mid-anterior region and positioned endovascularly in the lateral and septal portions of the coronary sinus.

FIG. 32I shows multiple pairs of electrodes 111, the pairs positioned endocardially (e.g. on an endocardial surface) high on the right atrial septum and positioned endovascularly in the Vein of Marshall.

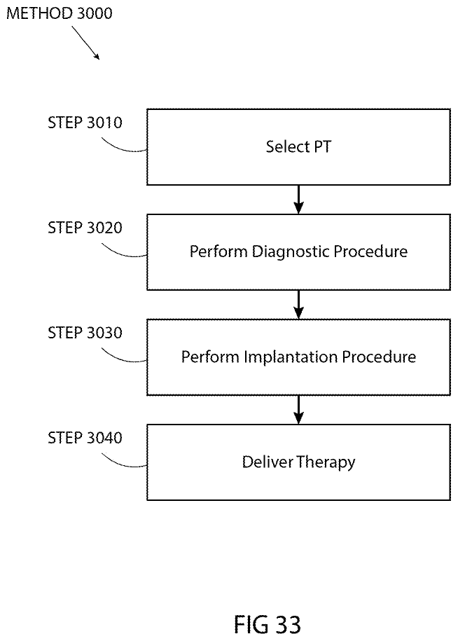

Referring now to FIG. 33, a flow chart of a method of treating a patient is illustrated, consistent with the present inventive concepts. Method 3000 describes selecting a patient to receive an implant, such as ID 100 described herein, and subsequently providing therapy to that patient. Method 3000 is described using the components of system 10, as described herein.

In Step 3010, a patient is selected. In some embodiments, the patient has a heart condition, such as a heart condition comprising: one, two, or more arrhythmias; ischemia; heart failure; and combinations of these. In some embodiments, the patient has atrial fibrillation, heart failure, or both.

In Step 3020, a first diagnostic procedure is performed. In the first diagnostic procedure, one or more locations for delivering stimulation energy can be selected (e.g. one or more locations for positioning electrodes for delivering stimulation energy can be selected).

In Step 3030, an implantation procedure is performed, where an ID 100 comprising one, two, or more implantable devices is implanted in the patient. The one or more implantable devices of ID 100 can be implanted within a chamber of the heart (e.g. on one or more endocardial surface locations), and/or outside the heart (e.g. on one or more epicardial surface locations).

In Step 3040, therapy is delivered to the patient, such as therapy comprising delivery of stimulation energy by an ID 100 comprising one, two, or more implanted devices. In some embodiments, stimulation energy is delivered by ID 100 in a continuous and/or intermittent basis. In some embodiments, stimulation energy is delivered “on demand”, such as when one or more sensors of system 10 detect an undesired condition, such as a current, and/or near-future (e.g. predicted) arrhythmia such as atrial fibrillation.

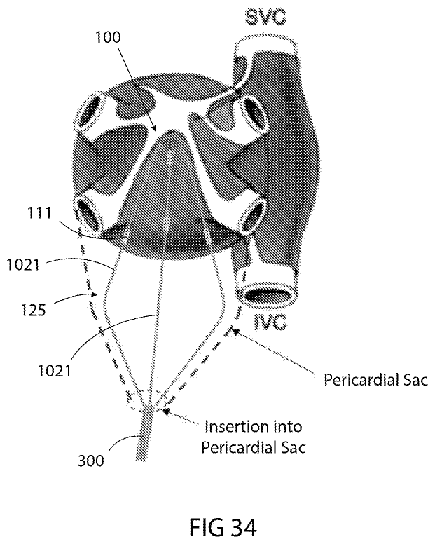

Referring now to FIG. 34, an anatomic view of an implantable device positioned on the epicardial surface of a heart is illustrated, consistent with the present inventive concepts. ID 100 of FIG. 34 can be similar to ID 100 described in reference to FIG. 1 and otherwise herein. In some embodiments, ID 100 comprises a geometry that leverages anatomical structures to anchor ID 100 when implanted onto the epicardial surface (e.g. between the epicardial surface and the pericardial sac), such as the geometry shown in FIG. 34. For example, the geometry of ID 100 can be configured to interact with anatomical structures (e.g. surfaces of anatomical structures) of an intended ID 100 implant location, such as locations on, within, and/or otherwise proximate one or more pericardial reflections, margins of the pericardial sac, and/or the pulmonary veins. As shown in FIG. 34, ID 100 can comprise geometry defined by one or more stabilizing elements, shafts 1021, where the geometry and mechanical properties of shafts 1021 are configured to leverage a resultant pressure applied by the pericardial sac to hold ID 100 against the epicardium. For example, ID 100 can be configured to interact with the top of a pericardial reflection as well as the pericardial margin at the apex of the ventricle, such as to stabilize (e.g. prevent migration of) ID 100 after implantation (e.g. at an inferior-superior position). The lateral margins of the pericardial sac along the ventricle, combined with the reflections at the pulmonary veins, can be used to stabilize ID 100, such as at a left lateral and right medial position. As described herein, shafts 1021 can comprise shape memory materials, such that these components of ID 100 can expand from a collapsed geometry (e.g. collapsed for implantation through clinician device 300), the expansion causing engagement of ID 100 with these particular portions of the anatomy. Also as described herein, an antenna, antenna 125 shown, can extend throughout shafts 1021 of ID 100, and/or one or more electrodes, electrodes 111 shown, can be positioned on shaft 1021.

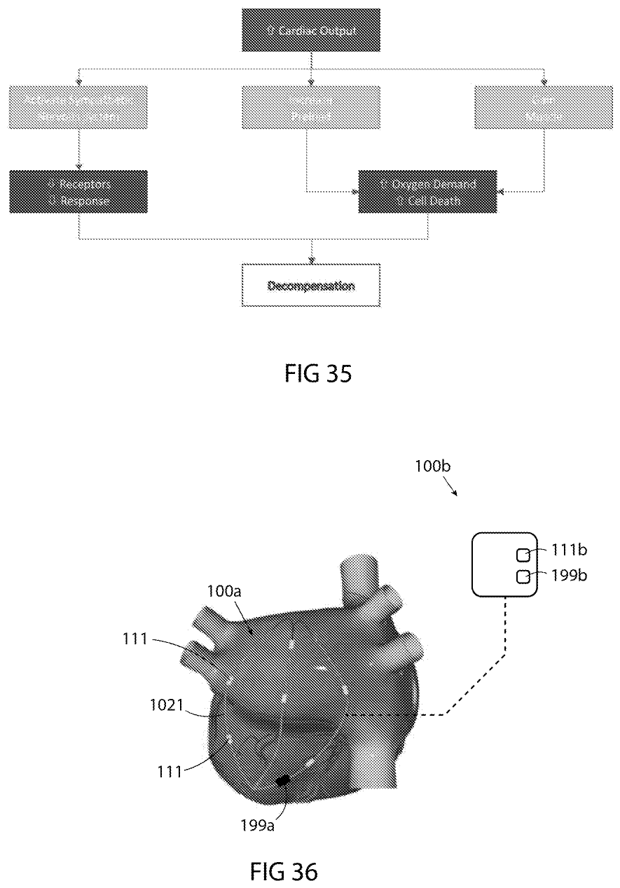

Referring now to FIG. 35, a flow chart of steps representing the heart's progression toward heart failure is illustrated, consistent with the present inventive concepts. Cardiac output is a multiplication of stroke volume and heart rate (Stroke Volume×Heart Rate=Cardiac Output). When the blood supply is not adequate, the heart compensates for these shortages by increasing the heart rate and/or the stroke volume. For example, the heart can compensate by activating the sympathetic nervous system to increase the heart rate, by increasing preload to add volume and contract harder, and/or by gaining muscle (“myocardial hypertrophy”) to achieve more forceful contractions. The sympathetic nervous system can prompt the heart to beat faster when an inadequate blood supply is detected. However, overactivation of the nervous system (receptor overuse) can result in downregulation of this compensatory response. In addition, the body can release specific hormones such as ADH or aldosterone which enables the ventricles to increase their filling volume. The increased volume or preload results in a more forceful contraction (higher pressure), which also increases cardiac output. However, this response requires more energy, which increases the demand for blood. When additional blood is not supplied to the myocardium, cardiac muscle cells begin to die. The heart can also gain muscle mass to enable more forceful contractions. In short, the surviving cardiac cells, called myocytes, become larger. As the cardiac myocytes grow, the chamber becomes proportionately smaller resulting in less volume, directly and negatively affecting cardiac output.

Heart failure clinical specialists use data to determine the state and progression of heart failure in a given patient. Several different scoring mechanisms (e.g. MAGGIC, NYHA, ACC/AHA, and GWTG) have been evaluated to give these physicians a frame of reference for each patient, but this data can require that several key symptoms, physiologic measurements, and bio markers be tracked on an ongoing basis. Some of these parameters can be monitored by the patients themselves, but this requires patient compliance, and others require routine tests by a lab. System 10 can be configured to collect these and/or other data, on a continuous and/or intermittent basis (e.g. hourly, daily, weekly). The data collected by system 10 can be used to identify a progression or even a regression in decompensation that would provide heart failure specialists with various clinical insights for managing the associated disease through pharmaceutical adjustments and/or other types of interventions.

In some embodiments, one or more sensors (e.g. functional element 199 comprising one or more sensors and/or one or more electrodes 111 configured as a sensor) of ID 100 (e.g. an ID 100 comprising one or more implantable devices) are positioned at one or more locations proximate heart tissue and are configured to produce signals from which a calculation of pressure within a chamber (e.g. pressure of the blood within the left atrium) can be determined (e.g. by one or more of algorithms 1005), such as is described in reference to FIG. 1 herein. In some embodiments, the signals are recorded from (e.g. ID 100 and the associated sensors are implanted at) a location outside of the chamber of the heart for which the chamber pressure is determined (e.g. outside of the left atrium when left atrial pressure is determined).

Referring additionally to FIG. 36, an anatomic view of an implantable device positioned on the epicardial surface of a heart is illustrated, consistent with the present inventive concepts. ID 100 of FIG. 36 can be similar to ID 100 described in reference to FIG. 1 and otherwise herein. In some embodiments, ID 100 is configured to perform key measurements and provide key data for a clinician to assess heart failure of a patient, such as the data described hereabove in reference to FIG. 35. As depicted in FIG. 36, electrodes 111 (e.g. two, three, four, or more electrodes) can be included in ID 100, for example, such that both the left atrium and left ventricle can adequately receive stimulation energy from these electrodes. When ID 100 is in a power state (e.g. charged via external power delivery), the data acquired by ID 100 can be provided to the clinician (e.g. either directly or through a cloud portal). There are several indicators that can be assessed with just the use of electrodes 111 alone.

For example, separation between the electrodes 111 can be determined and tracked over time, such as to assess heart failure of the patient. Since the stimulation falls off as a factor of distance (1/d for unipolar energy delivery and 1/d² for bipolar energy deliver), the timing between the stimulation signal and sensed events on other electrodes 111 can be used to calculate distance. Increases in distance can be an indicator for muscle stretch (e.g. muscle tissue stretch) or muscle growth. This technique can be applied to the left ventricle, the left atrium, or a combination of the two. Alternatively or additionally, sub-threshold pulses (e.g. pulses that are small enough in amplitude such that they do not stimulate) can be delivered, such as sub-threshold pulses that are delivered between pairs of electrodes from which the impedance of the subtended tissue can be calculated. This calculated impedance increases with stretch of the tissue, as compared to a baseline, unstretched state.

Alternately or additionally, pacing from one or more (e.g. all) of the electrodes 111, and determining (e.g. rapidly determining) the evoked response of the surrounding tissue, can be used to assess heart failure. For example, recording and/or assessing changes in the evoked response over time, can be indicative of decompensation.

Alternately or additionally, voltages and/or electric currents can be applied to electrodes 111, and the associated electric currents or voltages, respectively, can be measured by system 10 (e.g. by ID 100). These measured values can be used to compute impedance (Z), such as to produce impedance data used to assess heart failure. Changes in impedance over time can be used to identify lack of motion (e.g. lack of sufficient heart wall motion), and/or to identify presence of cellular necrosis.

An internal pulse generator (IPG) (a second ID 100, such as ID 100b) which is also implanted in the patient (e.g. subcutaneously implanted in the upper chest, and/or on the left or right lower abdomen), can also be used in conjunction with the first implant (e.g. an ID 100a) to record and/or provide additional data, such as data used to assess heart failure.

A functional element (e.g. functional element 199) comprising an accelerometer, positioned on and/or within an ID 100 can be used to track patient activity level, which can be used to assess heart failure. For example, detection of reduced activity level by system 10 can correlate to increased lethargy, which can be an indicator of decompensation.

Electrodes 111 (e.g. one, two, three, four, or more electrodes 111a and/or 111b) placed on or near the ID 100 can be configured to provide baseline ECG measurements, changes in heart rate and pulse morphology (e.g. long Q, ST elevation, decrease in heart rate variability).

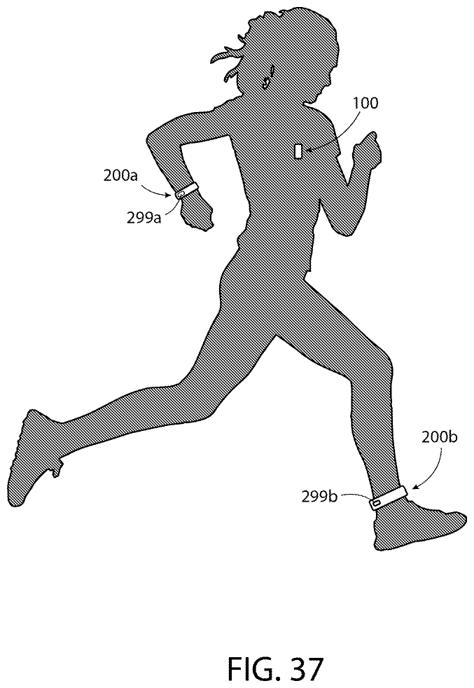

Referring now to FIG. 37, an anatomic view of a patient wearing external devices is illustrated, consistent with the present inventive concepts. ID 100 and EPDs 200 of FIG. 37 can be of similar construction and arrangement as similar devices described in reference to FIG. 1 and otherwise herein. In some embodiments, EPD 200 comprises an ankle worn device, EPD 200b shown. EPD 200b can be configured to monitor one or more patient parameters, such as via functional element 299b positioned proximate the patient's ankle. For example, functional element 299b can comprise a sensor configured to measure water accumulation in the ankle region (e.g. water accumulation associated with ankle oedema). Alternatively or additionally, functional element 299b can comprise a heartrate monitor, such as to provide data related to the patient's heart rate. Alternatively or additionally, functional element 299b can comprise one, two, or more sensors configured to measure the pulse of the patient (e.g. the pulse present in the ankle), such as to provide data related to the strength of the pulse present in the patient's ankle. In some embodiments, EPD 200a (a wrist worn device as shown) and/or 200b can comprise a fitness tracker, such as when functional elements 299a,b comprise one or more accelerometers and/or other sensors configured to provide data related to activity of the patient. In some embodiments, EPD 200 comprises a wearable device, such as a sock comprising one or more sensors (e.g. functional element 299 comprising one or more sensors). In some embodiments, EPD 200 comprises a flexible and/or stretchable material, such as to limit patient restriction caused by wearing EPD 200. In some embodiments, EPD 200 (e.g. at least a portion of EPD 200) is configured to expand and/or contract, such as to maintain contact with the skin of the patient (e.g. around the ankle of the patient) without restricting the patient, for example without restricting blood flow (e.g. without restricting blood flow of the ankle, such as if the ankle were to swell throughout the day).

Currently, appropriate management of chronic heart failure requires a considerable amount of participation by patients. Data related to changes in symptoms and disease progression are important in determining the optimal treatment for the heart failure patient. Key parameters to be monitored are changes in weight and sodium intake. Sodium can cause fluid retention and higher blood volume, which can place extra strain on an already weakened heart muscle. Additionally, sodium can interfere with the ability of any prescribed diuretics to work effectively.

Current methods for measuring the level of sodium intake and its effect (e.g. ankle oedema) are very limited. Chronic fluid accumulation is responsible for a substantial number of hospital admissions, and identifies patients with a worse prognosis than those admitted due to a sudden increase in LV filling pressures. Peripheral congestion in patients with heart failure usually develops over weeks or even months, and patients may present ‘acutely’ having gained an excess amount of fluid (e.g. over 20 liters of excess fluid, and hence over 20 kg of excess weight). The aim of clinical management of these patients is to remove the excess fluid, so that the patient is no longer congested when they leave hospital, now transitioning to a diagnosis of ‘chronic HF (CHF)’.

However, for many patients, some degree of congestion remains even with treatment, and it is not clear how many patients with CHF have subclinical congestion (e.g. have an excess of body fluid falling short of the volume required to cause overt peripheral oedema).

The development of peripheral oedema in patients with HF is related to fluid excess. As the heart starts to fail, renal perfusion falls. The kidneys respond by increasing the production of renin, leading to more aldosterone production, which is consequently followed by sodium and water retention. Arginine vasopressin (AVP) is also released, further enhancing fluid retention and stimulating thirst.

The activation of the renin-angiotensin-aldosterone and AVP systems maintain cardiac preload (more fluids) and afterload (vasoconstriction, mainly due to angiotensin II), thereby maintaining the homeostasis of the cardiovascular system but at a cost of increased systemic venous pressure (VP). The heart itself tends to worsen with time as the failing LV tends to dilate, as does the left atrium, particularly if mitral regurgitation develops. The elevated VP can further reduce renal blood flow as the gradient between mean renal arterial pressure (often itself decreased by the HF process) and VP declines. Glomerular filtration rate falls, enhancing and perpetuating the vicious cycle

Also, as peripheral oedema in patients with HF rises, their desire to exercise goes down. Exercise is a critically important activity for heart failure patients. Physical activity for most people (even those who have existing cardiovascular disease) is 30 minutes a day, 5 days a week. For heart failure patients, this may require pacing activity because of fatigue, but doing three 10-minute walks a day is still 30 minutes of activity.

System 10 can be configured to record, measure, assess, and/or determine these metrics (e.g. the values of one or more of the above parameters associated with heart failure or other heart condition). These metrics can be calibrated by system 10 by absolute measures, measured clinically as “baseline” measures. With calibration from baseline, relevant changes (e.g. clinically relevant changes) can be tracked, recorded, stored, and/or reported (e.g. reported to a clinician), such as on a continual and/or intermittent basis. System 10 can be configured to recalibrate these metrics (e.g. if needed), such as from absolute clinical measures taken in a follow-up procedure performed on the patient. In this arrangement, system 10 provides a quantitative methodology of patient monitoring that is clinically relevant in heart failure and/or other cardiac conditions (e.g. atrial fibrillation and/or another arrhythmia).

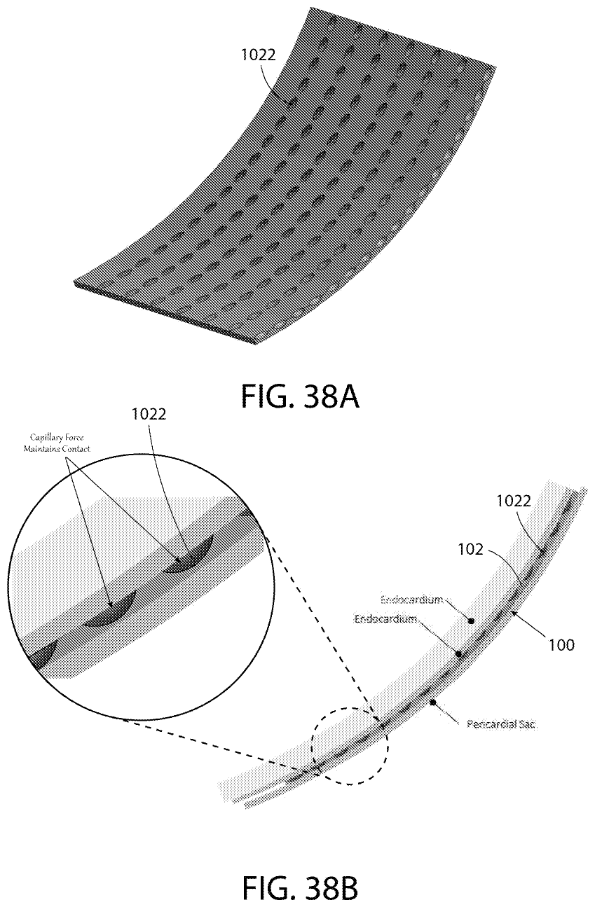

Referring now to FIGS. 38A and 38B, a perspective view of a substrate and a sectional anatomic view of a substrate including a magnified portion of the sectional view are illustrated, consistent with the present inventive concepts. As shown in FIG. 38A, substrate 102 of ID 100 can comprise an array of one or more capillary channels, channels 1022 shown. Channels 1022 can be configured to allow capillary action to occur between the epicardial wall and substrate 102, such as to secure (e.g. anchor) ID 100 to the epicardial wall. ID 100 of FIGS. 38A and 38B can be of similar construction and arrangement to ID 100 described in reference to FIG. 1 and otherwise herein. ID 100 (e.g. including channels 1022) can be positioned between the epicardium and the pericardial sac as shown in FIG. 38B.

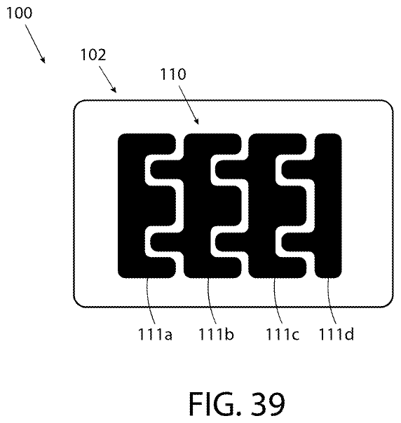

Referring now to FIG. 39, a side view of an electrode array positioned on a substrate of an implantable device is illustrated, consistent with the present inventive concepts. ID 100 of FIG. 39 can be of similar construction and arrangement to ID 100 described in reference to FIG. 1 and otherwise herein. Electrode array 110 can be positioned on substrate 102 of ID 100. Electrode array 110 can include one or more electrodes 111, such as electrodes 111a-d as shown (electrical connections to electrodes not shown for illustrative clarity). Electrodes 111a-d can each comprise a serpentine geometry and/or an interdigitated arrangement, each as shown. The serpentine geometry of each electrode 111 increases the “edge-length” of each electrode (e.g. compared to a rectangular electrode comprising a similar surface area). Increased edge-length can reduce electrode impedance and/or more evenly distribute stimulation current density in proportion to the increase in edge-length over the perimeter edge-length of a rectangular electrode subtending the same overall area (e.g. the increase in edge-length can be at least 2 times, at least 10 times, at least 50 times longer than the perimeter edge-length of a rectangular electrode subtending the same overall area). In some embodiments, one, multiple, and/or all of electrodes 111 can each comprise a footprint (e.g. the convex hull of the perimeter of the electrode) of less than 25 mm², such as less than 10 mm², such as less than 1 mm². In some embodiments, one, multiple, and/or all of electrodes 111 can each comprise a total edge length of at least 4 mm, such as at least 10 mm, such as at least 40 mm. In some embodiments, one, multiple, and/or all of electrodes 111 can each comprise a coating, such as a coating configured to lower the impedance of the electrode. In some embodiments, one, multiple, and/or all of electrodes 111 can each comprise an input impedance of less than 10 k ohms, such as less than 5 k ohms, such as less than 100 ohms. In some embodiments, one or more electrodes 111 are configured as sensing electrodes, and one or more electrodes 111 are configured as pacing electrodes. In some embodiments, electrodes 111 configured for pacing are configured differently from electrodes 111 configured for sensing, for example when pacing electrodes 111 comprise a different size, shape, coating, and/or impedance.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for providing therapy to a heart of a patient, the system comprising:
an implantable device configured to be implanted proximate the heart of the patient, the implantable device comprising:
an anchoring element configured to maintain the position of the implantable device after implantation in the patient;
at least one sensing electrode configured to sense electrical activity of the heart; and
at least two pacing electrodes configured to deliver electrical stimulation energy to tissue of the heart;
a communication device comprising a transceiver configured to transmit energy to the implantable device, and
a controller including one or more algorithms, wherein the one or more algorithms are executable to determine parameters of the stimulation energy to be delivered.

2-61. (canceled)
